(19) United States
(12) Patent Application Publication
(10) Pub. No.: US 2005/0192761 A1
(43) Pub. Date: Sep. 1, 2005

(54) Physiological profiling

(76) Inventors: Howard J. Jacob, Brookfield, WI (US); Peter J. Tonellato, Shorewood, WI (US); Nicholas J. Schork, San Diego, CA (US); Allen W. Cowley, Elm Grove, WI (US)

Correspondence Address: QUARLES & BRADY LLP, 411 E. WISCONSIN AVENUE, SUITE 2040, MILWAUKEE, WI, 53202-4497, US

(21) Appl. No.: 11/031,322

(22) Filed: Jan. 7, 2005

Related U.S. Application Data
(62) Division of application No. 09/960,234, filed on Sep. 20, 2001, now abandoned.
(60) Provisional application No. 60/234,023, filed on Sep. 20, 2000.

(51) Int. Cl. C12Q 1/70; G06F 19/00; G01N 33/48; G01N 33/50

(52) U.S. Cl. 702/19

(57) ABSTRACT

A new analytical strategy, termed physiological profiling, was developed that can capture physiological baselines and reveal relationships between particular phenotypes as a function of genotype in complex disease conditions. Physiological profiling offers a powerful strategy to visualize complex physiological processes. Combined with developing statistical analysis, this analytical tool is likely to facilitate our understanding of the biology of an organism.

### CROSS REFERENCE TO RELATED APPLICATIONS

This application claims priority from U.S. Provisional Application Ser. No. 60/234,023, filed on Sep. 20, 2000.

### STATEMENT AS TO FEDERALLY SPONSORED RESEARCH

Funding for the work described herein was provided by the federal government, which may have certain rights in the invention. This work was supported by National Heart, Lung, and Blood Institute Grant 1P50-HL-54998.

### BACKGROUND

1. Technical Field

The invention relates to methods and materials involved in identifying relationships among physiological determinants (parameters) associated with complex physiological processes that contribute to normal and pathological states of an organism.

2. Background Information

Genetic studies of complex multifactorial diseases such as asthma, hypertension, non-insulin-dependent diabetes mellitus (NIDDM), and insulin-dependent diabetes mellitus (IDDM) remain challenging due to heterogeneity in the clinical presentation of these diseases among patient populations. In addition, the modest contribution of each gene and/or the study of phenotypes that are distant from these gene effects, or both, have made identifying genes involved in these diseases difficult. Difficulties in elucidating the genetic basis of multifactorial diseases have become apparent from results obtained from total genome scans for quantitative trait loci (QTL) associated with asthma, hypertension, NIDDM, and IDDM in diverse human populations. (See Bleecker et al. (1997) Am J Respir Crit. Care Med. 156:S113-6; Julier et al. (1997) Hum Mol Genet 6:2077-85; Krushkal et al., (1999) Circulation 99:1407-1410; Hanis et al. (1996) Nat. Genet 13:161-166; and J. A. Todd (1995) Proc Natl Acad Sci USA 92, 8560-8565).

Furthermore, although genome-wide scans directed at the genetic basis of hypertension in rats have identified rough locations of genes on almost every rat chromosome, with loci confirmed on chromosomes 1, 2, 3, 5, 10, and 13 (J. P. Rapp (2000) Physiol. Rev. 80:135-172), no actual genes have been identified. The need for improved analytical tools, in addition to better phenotyping protocols, for identifying genes influencing complex phenotypes has been well articulated by Nadeau and Frankel (Nadeau et al. (2000) Nat. Genet. 25, 381-384).

### SUMMARY

The invention provides methods and materials related to identifying relationships among physiological traits—herein referred to as “physiological determinants.” More specifically, the invention provides a new analytical procedure for identifying relationships among physiological determinants associated with complex physiological processes that contribute to normal and pathological states of an organism. The analytical procedure, termed “physiological profiling,” involves, in broad form, three steps. First, a set of physiological determinants is identified. Second, correlation values are determined between pairs of physiological determinants for all possible pairs within the set. Third, the correlation values are organized into a clustered correlation matrix by organizing the corresponding physiological determinants along the axes of the matrix using a clustering method. From the resulting “physiological profile,” relationships between determinants can be identified. Physiological profiling can be used to characterize physiological processes in normal and diseased organisms. Results of physiological profiling can be used to classify diseased and/or normal organisms into groups based on correlation patterns determined. Physiological profiling also can be used in conjunction with genetic linkage analysis or gene expression profiling for functional genomics studies or clinical diagnosis.

In one embodiment, the invention provides a method of identifying relationships among physiological determinants within a set of physiological determinants. The method involves (1) determining a correlation value between two physiological determinants for all possible pairs of physiological determinants within the set; (2) constructing a correlation matrix using the determined correlation values; (3) constructing a clustered correlation matrix from the correlation matrix by clustering physiological determinants using a clustering method, and (4) identifying relationships among physiological determinants from the clustered correlation matrix. The clustering method can be based on known physiological relationships, known genetic linkages, or gene expression profiles. Alternatively, the clustering method can be a statistical method that does not rely on known physiological relationships, genetic linkages, or gene expression profiles.

In another embodiment, the method can involve constructing a colored clustered correlation matrix using a plurality of colors such that each color indicates a selected degree of correlation. The patterns of colors in the clustered correlation matrix can be used to identify physiological relationships.

In another embodiment, the set of physiological determinants can include at least 10, 20, or 50 determinants.

In another embodiment, the first member of each pair of physiological determinants can be derived from an individual and the second member of each pair of physiological determinants is the mean of physiological determinants from a population of individuals; and the correlation value is determined by a method that includes measuring the difference between the first member and the second member.

In another embodiment, the invention provides a method of assessing the physiological response of an organism to a challenge. The method includes a first, second, and third step. The first step involves constructing a first clustered correlation matrix using a set of physiological determinants. The first set of correlation values for all pairs of determinants in the set is obtained prior to the challenge. The second step involves constructing a second clustered correlation matrix using the same set of physiological determinants, and the second correlation values for all pairs of determinants in the set are obtained during or subsequent to the challenge. The third step involves comparing the first and second clustered correlation matrices to assess the physiological response of the organism to the challenge. The challenge can be, for example, a drug administration, an allelic substitution, or an environmental stressor.

In one embodiment, the correlation values in the matrices are represented by a plurality of colors, each color indicating a selected degree of correlation. In another embodiment, multiple clustered correlation matrices can be compared by comparing the patterns of colors of each matrix.

In another embodiment, the invention provides a method of assessing the change in physiological state of an organism or organisms over time. This method includes a first, second, and third step. The first step involves constructing a first clustered correlation matrix using a set of physiological determinants. The correlation values for all pairs of determinants in the set are obtained at a first time point. The second step involves constructing a second clustered correlation matrix using the same set of physiological determinants. The correlation values for all pairs of determinants in the second step are obtained at a second time point. The third step is comparing the first and second clustered correlation matrices to assess the change in physiological state of the organism from the first to the second time point. In another embodiment, more than two time points can be compared in this manner. The correlation values can be represented by a plurality of colors with each color indicating a selected degree of correlation. The clustered correlation matrices are compared by comparing the patterns of colors in the clustered correlation matrices.

In another embodiment, the invention provides a method of partitioning organisms into homogeneic subclasses. The method involves comparing the physiological profiles of the organisms and then partitioning the organisms into homogeneic subclasses based on differences in the physiological profiles. In one embodiment, expression profiling can be used to further partition the organisms into additional homogeneic subclasses based on expression profiling results. In another embodiment, the organisms can exhibit a multifactorial disease condition.

In another embodiment, the invention provides a method of assigning an organism to a homogeneic subclass of organisms. The method includes generating a physiological profile of the organism and identifying the organism as belonging to a homogeneic subclass based on the physiological profile. In another embodiment, the homogeneic subclass of organisms exhibits a multifactorial disease condition.

In another embodiment, the invention provides a method of determining the contribution(s) of a gene or genes to a physiological process in an organism. The method involves a first, second, third, and fourth step. The first step involves generating a first expression profile and a first physiological profile of the organism before a challenge. The second step involves generating a second expression profile and a second physiological profile of the organism during or after the challenge. The third step involves comparing the first expression profile and first physiological profile with the second expression profile and second physiological profile. The gene or genes are identified by the difference or differences in the first and second expression profiles. The physiological contributions of the same gene or genes are indicated by changes in the first and second physiological profiles.

In another embodiment, the invention provides a method of determining the contribution(s) of a gene or genes to a physiological process in an organism. The method involves a first, second and third step. The first step is generating a first expression profile and a first physiological profile of the organism at a first time. The second step is generating a second expression profile and a second physiological profile of the organism at a second time. The third step is comparing the first expression profile and first physiological profile with the second expression profile and second physiological profile. The gene or genes are identified by the difference or differences in the first and second expression profiles. The physiological contributions of the gene or genes are indicated by changes in the first and second physiological profiles.

In another embodiment, the invention provides a computer-readable medium that includes a physiological profile. In another embodiment, the physiological profile has a plurality of colors, each color indicating a selected degree of correlation.

In another embodiment, the computer-readable medium can have stored computer-readable instructions for performing the above-described methods.

In another embodiment, the invention provides a method of determining whether a hypertensive patient is a modulator or non-modulator. The method involves determining the allelic status of a gene encoding renin in the patient. The allelic status of a patient is determined by identifying which allele of a relevant gene, among various possible alleles of that gene, is possessed by that patient.

In another embodiment, the invention provides a method of determining whether a patient is at risk for hypotension following administration of a vasoconstrictor agent. The method involves determining the allelic status of a gene encoding NOSII in the patient.

In another embodiment, the invention provides a method of determining whether a patient is at risk for hypotension following administration of a vasoconstrictor agent. The method involves determining the allelic status of a gene encoding NOSIII in the patient.

In another embodiment, the invention provides a method for modifying or supplementing actuarial tables for life and health insurance. The method involves identifying homogeneic subclasses of organisms, e.g. humans, as described earlier, and modifying or supplementing actuarial tables based on the identified homogeneic subclasses.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a comprehensive linkage map of 81 determinant phenotypes (96 QTL) in the autosomal genome of F2 male progeny (n=113) from an SS/JrHsd/Mcw and BN/SsNHsd/Mcw intercross. Vertical bars on the left side represent the 95% confidence intervals (CI) of individual QTL. Green bars indicate CI from parametric analysis, while orange bars indicate CI from non-parametric analysis. Phenotype designations and peak LOD scores (green=parametric) and Z-scores (orange=non-parametric), respectively, are presented on the right of each chromosome.

FIG. 2 is a randomized colored correlation matrix of BN phenotypes. Strong positive correlations are represented in red, strong negative correlations are blue, while low correlations are in gray and black.

FIG. 3 is a physiological profile of BN phenotypes ordered by functional clustering using Guyton's model of blood pressure control. Strong positive correlations are represented in red, strong negative correlations are blue, while low correlations are in gray and black.

FIG. 4 is a composite matrix of two physiological profiles, one generated using functional clustering and the second generated using purely statistical clustering. Strong positive correlations are represented in red, strong negative correlations are blue, while low correlations are in gray and black.

FIG. 5 is two physiological profiles consisting of phenotypes associated with regulation of blood flow for parental BN and SS rats. Strong positive correlations are represented in red, strong negative correlations are blue, while low correlations are in gray and black.

FIG. 6 is two composite physiological profiles of parental BN and F2 progeny rats generated by overlaying functionally clustered correlation matrices with algorithm clustered correlation matrices.

FIG. 7A is a comparison of the physiological profile of all F2 progeny rats with the physiological profile of progeny rats that fall in the left 10% tail of a distribution after a salt challenge. Strong positive correlations are represented in red, strong negative correlations are blue, while low correlations are in gray and black.

FIG. 7B is a comparison of the physiological profile of all F2 progeny rats with the physiological profile of progeny rats that fall in the right 10% tail of a distribution after a salt challenge. Strong positive correlations are represented in red, strong negative correlations are blue, while low correlations are in gray and black.

FIG. 8A is a physiological profile of phenotypes associated with arterial blood pressure in F2 male rats homozygous SS for D10Mgh14 (NOSII gene region). Strong positive correlations are represented in red, strong negative correlations are blue, while low correlations are in gray and black. The expanded insert represents correlations among blood pressures determined immediately before, during, and after administration of norepinephrine, angiotensin II, and acetylcholine.

FIG. 8B is a physiological profile consisting of phenotypes associated with arterial blood pressure in F2 male rats homozygous BN for D10Mgh14 (NOSII gene region). Strong positive correlations are represented in red, strong negative correlations are blue, while low correlations are in gray and black. The expanded insert represents correlations among blood pressures determined immediately before, during, and after administration of norepinephrine, angiotensin II, and acetylcholine.

FIG. 9A is a graph illustrating the correlation between mean arterial pressure before and after infusions of norepinephrine in F2 rats homozygous SS (open circles) for the NOSII gene and those homozygous BN (closed circles) for the NOSII gene.

FIG. 9B is a bar graph summarizing the average levels of mean arterial pressure before (solid bars) and following completion (open bars) of the intravenous infusions of three doses of norepinephrine in male rats carrying the SS or BN allele at NOSII.

FIG. 10 is two physiological profiles of French Canadian and African American hypertensive patients. Strong positive correlations are represented in red, strong negative correlations are blue, while low correlations are in gray and black.

### DETAILED DESCRIPTION

The invention provides methods and materials related to identifying relationships among physiological traits—herein referred to as “physiological determinants.” More specifically, the invention provides a new analytical procedure for identifying relationships among physiological determinants associated with complex physiological processes that contribute to normal and pathological states of an organism. The analytical procedure, termed “physiological profiling,” involves, in broad form, three steps. First, a set of physiological determinants is identified. Second, correlation values are determined between pairs of physiological determinants for all possible pairs within the set. Third, the correlation values are organized into a clustered correlation matrix by organizing the corresponding physiological determinants along the axes, for example, top, bottom, or sides of the matrix using a clustering method. From the resulting “physiological profile,” relationships between determinants can be identified. As used herein, the term “physiological profile” refers to a clustered correlation matrix generated using (1) a set of physiological determinants, ordered using a clustering method, and (2) the correlation values determined for all possible pairs of physiological determinants in the set. As used herein, the term “physiological profiling” refers to an analytical procedure involving (1) identifying a set of physiological determinants, (2) determinating correlation values for all possible pairs of determinants with the set, and (3) generating a clustered correlation matrix by organizing the correlation values into a matrix using a clustering method that orders the determinants in a non-random fashion along the axes of a matrix. Physiological profiling can be used to characterize physiological processes in normal and diseased organisms. Results of physiological profiling can be used to classify diseased and/or normal organisms into groups based on correlation patterns determined. Physiological profiling also can be used in conjunction with genetic linkage analysis or gene expression profiling for functional genomics studies or clinical diagnosis.

Physiological Determinants

The first step in generating a physiological profile is identification of a set of physiological determinants. As used herein, the term “physiological determinants” refers to physiological traits that can be determined experimentally or derived from experimentally measured data. For example, a measured physiological determinant can be weight (e.g. weight of an organism or an organ such as a kidney), volume (e.g. urine volume), or blood pressure (e.g. diastolic or systolic blood pressure). A derived physiological determinant can be, for example, mean blood pressure, standard deviation of mean arterial pressure, or the difference between (1) blood flow/gram of kidney weight after administration of a drug and (2) control blood flow per gram of kidney weight.

Physiological determinants can be obtained before, during, or after a challenge. A challenge can be any condition or event that triggers a physiological response or alters homeostasis. The challenge can be, for example: a disease condition, one or more allelic substitutions, an environmental stressor (e.g. hypoxia, high salt intake), contact with a naturally- or non-naturally occurring chemical or macromolecule, infection by a biological material (e.g. bacteria, viruses, prions), and the presence or absence of exercise.

One example of a derived physiological determinant is “delta renal blood flow from Angiotensin II dose 2 minus control renal blood flow.” In this example, the physiological determinant is derived by subtracting renal blood flow determined before a challenge, from delta renal blood flow determined after a challenge. The challenge is Angiotensin II.

Physiological determinants reflect the status of the relevant complex physiological system, for which the determinants serve as estimates of biological function. Complex physiological systems include, without limitation, the respiratory system, cardiovascular system, nervous system, digestive system, endocrine system, immune system, lymphatic system, renal system, skeletal system, catabolic and metabolic systems, and the digestive system.

Physiological determinants can include coronary determinants associated with mechanical, electrical, and biochemical functions in the heart, and with the heart's ability to resist ischemia. Examples include, without limitation, ischemic peak contracture (mmHg), ischemic time to onset of contracture (sec), ischemic time to peak contracture (sec), post-ischemic coronary flow rate (mL/min), enzyme leakage (IU/g wet weight), heart rate (beats/min), infarct size (% LV), left ventricle developed pressure (mmHg), left ventricle diastolic pressure (mmHg), left ventricle systolic pressure (mmHg), recovery coronary flow rate (% recovery), recovery developed pressure (% recovery), recovery heart rate (% recovery), recovery systolic pressure (% recovery), coronary flow rate (ml/min/g), enzyme leakage (IU/g wet weight), post-ischemic heart rate (beats/min), pre-ischemic heart wet weight (g), left ventricle developed pressure (mmHg), left ventricle diastolic pressure (mmHg), and pre-ischemic left ventricle systolic pressure (mmHg).

Physiological determinants can be associated with the vascular system and include vascular responsiveness to acute vasoconstrictors and dilators, vascular function, and the susceptibility to developing injury in response to a high salt diet. Examples include, without limitation, dilator response to acetycholine EC₅₀ (1×10⁻⁷ mole), dilator response to acetycholine Log EC₅₀ (Log molar), fast slope of phenylephrine-induced contraction (gram/min), maximum force (g) per wet weight of aorta (gram/min), % maximum relaxation acetylcholine (%), % maximum relaxation of phenylephrine-induced contraction by 0% O₂ (%), % maximum relaxation of phenylephrine-induced contraction by 10% O₂ (%), % maximum relaxation of phenylephrine-induced contraction by 5% O₂ (%), % maximim relaxation sodium Nitroprusside (%), constrictor response to phenylephrine EC₅₀ (1×10⁻⁷ mole), constrictor response to phenylephrine Log EC₅₀ (Log molar), dilator response to sodium nitroprusside EC₅₀ (1×10⁻⁷ mole), dilator response to sodium nitroprusside Log EC₅₀ (Log molar), and slow slope of phenylephrine-induced contraction (gram/min).

Physiological determinants can be associated with renal function such as blood pressure responsiveness to acute vasoconstrictors and dilators, and renal tubular function and susceptibility to developing renal injury in response to high salt diets. Examples include, without limitation, baseline HR for AngII dose-response relationship (beats/min), NE dose-response relationship (beats/min), baseline MAP for AngII dose-response relationship (mmHg), and baseline MAP for NE dose-response relationship (mmHg). Examples also include high salt creatinine clearance (mL/min), low salt creatinine clearance (mL/min), delta HR to 10 ng/kg/min AngII (beats/min), delta HR to 0.2 ug/kg/min NE (beats/min), delta HR to 25 ng/kg/min AngII (beats/min), delta HR to 0.5 ug/kg/min NE (beats/mD), delta HR to 50 ng/kg/min AngII (beats/mm), delta HR to 1.0 ug/kg/min NE (beats/min), delta HR to 5 ng/kg/min AngII (beats/min), and delta HR to 0.1 ug/kg/min NE (beats/min). Examples also include change in heart rate with salt depletion (beats/min), high salt heart rate (beats/min), low salt heart rate (beats/min), pre- to post-control delta HR following ANGII (beats/min), pre to post control delta HR following NE (beats/min), delta MAP to 10 ng/kg/min AngII (mmHg), delta MAP to 0.2 ug/kg/min NE (mmHg), delta MAP to 25 ng/kg/min AngII (mmHg), delta MAP to 0.5 ug/kg/min NE (mmHg), delta MAP to 50 ng/kg/min AngII (mmHg), delta MAP to 1.0 ug/kg/min NE (mmHg), delta MAP to 5 ng/kg/min AngII (mmHg), delta MAP to 0.1 ug/kg/min NE (mmHg), change in mean arterial pressure with salt depletion (mmHg), high salt mean arterial pressure (mean of three days of high salt pressure recordings) (mmHg), low salt mean arterial pressure (one day of recording following salt depletion) (mmHg), pre- to post-control delta MAP following ANGII (mmHg), pre- to post-control delta MAP following NE (mmHg), high salt plasma creatinine (mg/dL), low salt plasma creatinine (mg/dL), change in plasma renin activity with salt depletion (ls-hs) (ng angl/mL/hr), high salt plasma renin activity (ng angl/mL/hr), low salt plasma renin activity (ng/mL/hr), high salt urinary excretion of sodium (mEq/day), low salt urinary excretion of sodium (mEq/day), high salt urine microalbumin excretion (mg/day), high salt urine osmolality (mOsm/L), low salt urine osmolality (mOsm/L), and high salt urine protein excretion (mg/day).

Physiological determinants also can be associated with lung functions such as airway methacholine sensitivity, pulmonary vascular mechanics, pulmonary endothelial angiotensin converting enzyme activity, and pulmonary endothelial redox status in normal and chronically hypoxic conditions. Examples include, without limitation, alpha, (a statistical measure of characterizing the white noise component of blood pressure −1/mmHg), body weight (kg), FAPGG metabolism-surface area product (mL/min×kg), lung dry weight/body weight ratio (g/kg), hematocrit (%), MB+metabolism-surface area product 1 (mL/min×kg), MB+MSAP 3 (mL/min×kg)/FAPGG MSAP (mL/min×kg), MB+metabolism-surface area product 3 (mL/min×kg), methacholine ED50 (mg/kg), right ventricle/Left ventricle weight ratio (w/w ratio), and r@flow=100 mL/min/g (“r” represents left ventricular resistance, mmHg×min×kg/ml).

Physiological determinants can be associated with respiration such as respiratory control mechanisms and the pattern of breathing and lung function in the conscious state under acute conditions of hypoxia, hypercapnia, and exercise. Examples include, without limitation, heart rate during control (co) HYPERCAPNIA (beats/min), heart rate during control (co) HYPOXIA (beats/min), change in heart rate from rest to run (delta re v rn) (beats/min), change in heart rate from rest to walk (delta re v wk) (beats/min), heart rate during minute 7 of hypercapnia (b2) (beats/min), heart rate during minute 7 of hypoxia (b2) (beats/min), heart rate treadmill resting 3 minute average (re) (beats/min), heart rate running 30 second average (rn) (beats/min), and heart rate walking 30 second average (wk) (beats/min). Examples also include mean arterial pressure during control (b1) HYPERCAPNIA (mmHg), mean arterial pressure during control (b1) HYPOXIA (mmHg), change in mean arterial pressure from rest to run (delta re v rn) (mmHg), change in mean arterial pressure from rest to walk (delta re v wk) (mmHg), mean arterial pressure during minute 7 of hypercapnia (b2) (mmHg), mean arterial pressure during minute 7 of hypoxia (b2) (mmHg), m an arterial pressure treadmill resting 3 minute average (re) (mmHg), mean arterial blood pressure treadmill 30 second average (rn) (mmHg), and mean arterial blood pressure treadmill 30 second average (wk) (mmHg). Examples also include control (co) PaCO₂ HYPOXIA (mmHg), change in PaCO₂ between Control (co) and hypoxia (h2) (mmHg), hypoxia (h2) PaCO₂ (mmHg), arterial PCO₂ at rest 30 second average (re) (mmHg), arterial PCO₂, rilnning 30 second average (rn) (mmHg), arterial PCO₂ walking 30 second average (wk) (mmHg), control (co) PaO₂ HYPOXIA (nimHg), change in PaO₂ between Control (co) and hypoxia (h2) (mmHg), hypoxia (h2) PaO₂ (mmHg), arterial PO₂ at rest 30 second average (re) (mmHg), arterial PO₂ running 30 second average (rn) (mmHg), arterial PO₂ walking 30 second average (wk) (mmHg), change in PCO₂ from rest to run (delta re v rn) (mmHg), and change in PCO₂ from rest to walk (delta re v wk) (mmHg). Examples also include control (co) pH HYPOXIA (pH), change in pH between Control (co) and hypoxia (h2) (pH), change in pH from rest to run (delta re v rn) (pH), change in pH from rest to walk (delta re v wk) (pH), hypoxia (h2) pH (pH), arterial pH at rest 30 second average (re) (pH), arterial pH running 30 second average (rn) (pH), arterial pH walking 30 second average (wk) (pH), change in PO₂ from rest to run (delta re v rn) (mmHg), and change in PO₂ from rest to walk (delta re v wk) (mmHg). Examples also include rectal temperature for control HYPERCAPNIA (° C.), rectal temperature for control HYPOXIA (° C.), change in rectal temperature from rest to post exercise (° C.), change between control and hypercapnic rectal temperature (° C.), change between control and hypoxic rectal temperature (° C.), rectal temperature following running on treadmill (° C.), rectal temperature after hypercapnia (° C.), rectal temperature after hypoxia (° C.), rectal temperature at rest (° C.), pulmonary ventilation (VE) control (co). HYPERCAPNIA (mL/min), and pulmonary ventilation (VE) control (co). HYPOXIA (mL/min). Examples also include % change from (co)_in ventilation to (h2) hypercapnia, % change from (co) in ventilation to (h2) hypoxia, pulmonary ventilation (VE) at hypercapnia from minute 2-3 (h1) (mL/min), pulmonary ventilation (VE) at hypoxia from minute 2-3 (h1) (mL/min), % change from (co)_in ventilation to (h2) hypercapnia, % change from (co) in ventilation to (h2) hypoxia, pulmonary ventilation (VE) at hypercapnia from minute 9-10 (h2) (mL/min), pulmonary ventilation (VE) at hypoxia from minute 9-10 (h2) (mL/min), breathing frequency (f) during (co) HYPERCAPNIA (breaths/min), breathing frequency (f) during (co) HYPOXIA (breaths/min), % change from (co) in frequency (f) under hypercapnia conditions to (h2), % change from (co) frequency (f) under hypoxic conditions to (h1), breathing frequency (f) at hypercapnia during (h1) (breaths/min), breathing frequency (f) at hypoxia during (h1) (breaths/min), % change from (co) in frequency (f) under hypercapnia conditions to (h2), % change from (co) in frequency (f) under hypoxic conditions to (h2), breathing frequency (f) at hypercapnia during (h2) (breaths/min), breathing frequency (f) at hypoxia during (h2) (breaths/min), tidal volume (VT) during (co) HYPERCAPNIA (ml), tidal volume (VT) during (co) HYPOXIA (mL), % change from (co) in Tidal volume (VT) under hypercapnia conditions to (h1), % change from (co) in tidal volume (VT) under hypoxic conditions to (h1), tidal volume (VT) at hypercapnia (h1) (mL), tidal volume (VT) at hypoxia (h1) (mL), % change from (co) in tidal volume (VT) under hypercapnia conditions to (h2), % change from (co) in tidal volume (VT) under hypoxic conditions to (h2), tidal volume (VT) at hypercapnia (h2) (mL), and tidal volume (VT) at hypoxia (h2) (mL).

Physiological determinants can include indices of clinical chemistry and hematology associated with normoxic and chronically hypoxic conditions in the serum or plasma of an organism such as a mammal. Examples include, without limitation, amounts of albumin (g/dL), alkaline phosphatase (U/L), alanine transaminase (ALT) (U/L), anion gap (mmol/L), aspartate transaminase (AST) (U/L), bicarbonate (mmol/L), calcium (mg/dL), chloride (mmol/L), cholesterol (mg/dL), creatinine (mg/dL), eosinophil (absolute counts in terms of 1000 cells/μL), globulin (g/dL), glucose (mg/DI), plasma hematocrit (%), hemoglobin (g/dL), lymph (absolute count in terms of 1000 cells/μL), mean corpuscular hemoglobin concentration (pg), mean corpuscular hemoglobin concentration (g/dL), mean corpuscular volume (fL), and amounts of monocytes (absolute count in terms of 1000 cells/μL), phosphorus (mg/dL), platelet count (in terms of 1000 cells/uL), potassium (mmol/L), red blood cell (1×10⁶/uL), segmented neutrophils (in terms of 1000 cells/μL), sodium (mmol/L), total bilirubin (mg/dL), total protein (g/dL), urea nitrogen (mg/dL), and white blood cell count (in terms of 1000 cells/μL).

Physiological determinants also can include histological characterization of tissues under various physiological conditions, for example, normoxic, hypoxic, or high or low salt conditions. Examples include, without limitation, general anatomical measurements, measurements derived from medical imaging modalities, or quantifications of biomarkers commonly used in disease diagnostics of various tissues, for example, those from the aorta, microvasculature, stomach, breast, testes, ovaries, bone, lymphocytes, heart, kidney, lung, intestinal, brain, liver, pancreas, and prostate.

Physiological determinants also can be specific to other disease conditions. For example, physiological determinants such as tumor size, cell type, tests of cell type, cell/tumor response to various agents, tumor location, primary site of tumor, secondary sites of tumors, genes associated with cancer, and microarray patterns of gene expression associated with each stage of cancer as well as those described above can be used to assess a cancer condition.

Correlation Values and Correlation Matrices

As used herein, the term “correlation value” refers to a mathematical relationship between two physiological determinants calculated using statistical methods. Standard mathematical and statistical methods can be used to determine correlation or other statistical or quantitative measures used to characterize relationships between two or more physiological determinants. Linear, polynomial, and multiple regression analysis, as well as covariance analysis, T-test, and mathematical (linear or non-linear functional relationships) are examples of methods that can be used to determine statistical or mathematical measures that quantitatively relating two or more determinants. Both parametric (model-based) analytical methods (e.g. Pearson correlation coefficient, regression methods, mathematical functional relationships) and non-parametric analytical methods (e.g. Spearman correlation coefficient, Z-scores, and Wilcoxian rank sum) can be used. To obtain a correlation value between two physiological determinants such as mean arterial pressure (MAP) and heart rate, for example, the MAP and heart rate for all individuals in a study are measured. From the measured values of MAP and heart rate, a correlation value, a quantitative measure of the relationship between MAP and heart rate, can be determined using the formula:

$${C}_{x y} = \frac{\sum ( {X}_{i} * {Y}_{i} )}{( SQRT [ \sum ( {X}_{i} * {X}_{i} ) * \sum ( {Y}_{i} * {Y}_{i} ) ] )}$$

where Xᵢ represents MAP, Yᵢ represents heart rate, and “i” can be 1 to “N”. “N” represents the size of the population being studied. For example, if 100 patients are used in the study, then N=100. The 100 values of MAP and 100 values of heart rate are presented as 100 pairs of (Xᵢ, Yᵢ). C_{xy} is the correlation value of the MAP and heart rate.

The correlation value between the two physiological determinants obtained using the above formula is the basis of assigning a color to the correlation matrix. The larger the number of determinants, the larger the correlation matrix, and the more quantification required. For M determinants, the matrix is size M, and the number of determinants to be calculated is M* M/2. In every case, the mathematical or statistical quantification can be normalized in such a way as to allow colorization in a consistent manner. All values, for example, are normalized into the range of −1 to 1. This allows for using the same non-numerical indications of degrees of correlation.

Other quantifications between any two determinants (Xᵢ, Yᵢ) can be used in the same manner as the correlation matrix. For example, if the relationship between the two determinants is non-linear, for example exponential, then the correlation measure would not provide the most accurate method in characterizing the relationship between X with Y. Rather, a mathematical model (Y=EXP(B*X) would be the best approach to characterize the relationship. In this case, the best-fit estimate (based upon a nonlinear regression between Y and X) of B would represent the quantified relationship. Hence, rather than correlation coefficient, the profile matrix would have the estimate of B in the cell representing the relationship between X and Y.

Any combination of correlation coefficients, best-fit estimates, or other appropriate measures of the relationships between the variables can be used. For convenience, all such measures are referred to herein as “correlation values” between the relevant physiological determinants. In some cases, more than one quantification may be needed to represent the relationship between two determinants. For example if Y=mX+b, linear regression would provide the two measures (m, b) representing the quantified relationship between Y and X. In this case, more than one “cell” of the profile matrix is required to represent the “profile” attributed to the two determinants (X and Y). Whatever the approach to extract quantified measures of the relationships between the determinants, in every case there is a simple approach to assigning those measures to the correlation matrix, assigning a color or other graphical representation to the measure in the matrix, and thus creating a colorized or other graphical representation of the physiological profile.

Correlation values can be any value between 1 and −1 inclusive. For example, correlation values can be −1, −0.99, −0.9, −0.88, −0.8, −0.77, −0.7, −0.66, −0.6, −0.55, −0.5, −0.44, −0.4, −0.33, −0.3, −0.22, −0.2, −0.11, −0.1, 0, 0.1, 0.11, 0.2, 0.22, 0.3, 0.33, 0.4, 0.44, 0.5, 0.55, 0.6, 0.66, 0.7, 0.77, 0.8, 0.88, 0.9, 0.99, 1, or any value in between.

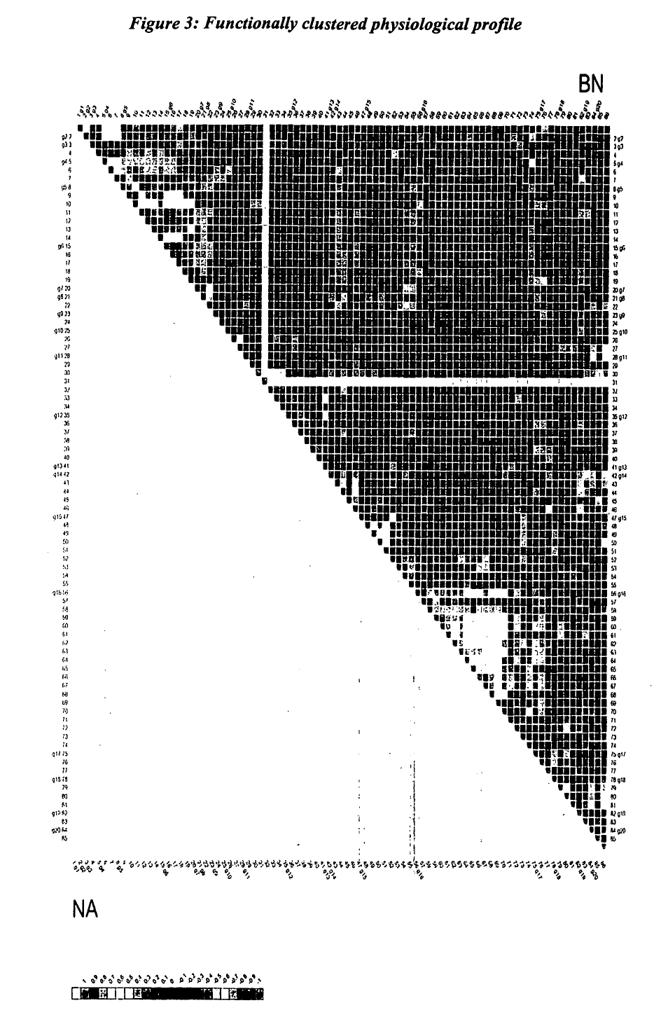

Once determined, con-elation values for all possible pairs of determinants within a set of determinants can be presented on a correlation matrix. A “set” of physiological determinants is a group of determinants that can be associated with a particular physiological condition. A correlation matrix can be depicted, for example, as a two-dimensional graph in which the determinants are ordered along the X and Y axes (e.g., sides, bottom, and top of a two-dimensional array; see, e.g., FIG. 3). Correlation values are placed in locations within the matrix equivalent to locations specified by particular coordinates (Xs, Ys). Determinants can be ordered, i.e. clustered, in a number of non-random ways using a clustering method. Determinants can be clustered using known physiological, biochemical, or functional relationships. For example, all determinants related to a particular biochemical pathway can be clustered next to each other, while all determinants related to a biological function, e.g. renal blood flow, can be clustered together. As used herein the term “functionally clustered” refers to the ordering of determinants based on known physiological, biochemical, or functional relationships. Determinants also can be clustered using purely statistical or mathematical methods involving models (parametric methods) or without models (non-parametric methods). Examples include, without limitation, hierarchial, self-organizing maps (SOMs), or principal component analysis. Standard statistical methods are described in Everitt, B. S. (1993) Cluster Analysis, 3rd Edition, Edward Arnold, Ltd., London, UK; SAS/STAT User's Guide (1990) Version 6, Fourth Edition, Volume 1, pages 519-614; and SAS/STAT User's Guide, 1990, Version 6, Fourth Edition, Vol 2, pages 1614-1631. As used herein, the term “algorithm clustering” refers to clustering determinants using a purely mathematical or statistical method. A correlation matrix in which the determinants are clustered using functional or statistical methods is herein referred to as a “clustered correlation matrix” or a “physiological profile.”

Correlation values can be presented on a clustered correlation matrix as numeric values. Correlation values also can be presented in any manner that facilitates visual interpretation. For example, a color scheme in which a particular color represents a particular degree of correlation can be used. Other types of designations effective in differentiating highly negative or positive, moderately negative or positive, or low correlation values also can be used, for example shading, stippling, or cross-hatching.

In contrast to the presentation of correlation values and relationships on a visible clustered correlation matrix/physiological profile, the generation of the physiological profile can be performed by a computer such that only differences in correlation structures under different conditions, or shifts in correlations determined from comparing profiles obtained in response to a challenge or over time, are reported to the experimenter. In this embodiment, determination of correlation values, statistical analyses, phenotypic clustering, and identification of correlation structures or shifts in correlations are performed in silico.

Applications

Physiological profiling is a method of capturing complex physiological processes that contribute to normal and pathological states of an organism. Since physiological profiling reveals relationships between physiological determinants, a physiological profile generated using determinants related to a complex physiological system can be used to capture the efficiency and status of that particular system.

In one embodiment, physiological profiling can be used to follow development of an organism over time. An organism can be subjected to physiological profiling at various points in its life cycle. The resulting physiological profiles can be correlated with other aspects of the organism's development such as physical, mental, and physiological development as well as aging. The resulting physiological profiles also can be correlated with the health status of an organism as well as with susceptibility to infections and development of disease conditions.

In another embodiment, physiological profiling can be performed for large populations. Resulting physiological profiles can be used in conjunction with, as replacements for, or as supplements to, existing actuarial tables. An individual's profile can be used for predicting life expectancy by linking with actuarial tables.

In another embodiment, physiological profiling can be used as a diagnostic method. For example, healthy organisms and those exhibiting, or predisposed to developing, a disease condition can be distinguished by physiological profiling based on differences in relationships, i.e. correlations, among mechanistically relevant physiological determinants. To distinguish a healthy organism from an organism having a disease condition, the physiological profiles of organisms or groups of organisms representative of normal and disease conditions are determined. The resulting physiological profiles representing a normal and a diseased condition can be used for diagnostic purposes.

In another embodiment, physiological profiling can be used to capture physiological states of multifactorial diseases. As used herein, the term “multifactorial disease” refers to a disease associated with multiple genetic loci as well as environmental factors. Examples of multifactorial diseases include, without limitation, obesity, hypertension, end stage renal disease, and growth defects. Multifactorial diseases also include heart conditions such as myocardial infarction, left ventricular hypertrophy, congestive heart failure; diabetes; cancers such as leukemia, lymphoma, and myeloma; autoimmune diseases such as lupus, multiple sclerosis, rheumatoid arthritis, type 1 diabetes mellitus, psoriasis, thyroid diseases, systemic lupus erythematosus, scleroderma, celiac disease/gluten sensitivity, and inflammatory bowel diseases; and mental illnesses such as schizophrenia, bipolar depression, and Parkinson's disease.

Typically, the patient population for a particular multifactorial disease, including those described above, is heterogeneic in that the clinical presentation of the disease condition varies among individuals of the population. Physiological profiling can be used to partition heterogeneity, i.e., to reduce the heterogeneous patient population exhibiting a multifactorial disease into more homogeneous subclasses of the multifactorial disease. As used herein, the term “homogeneic subclass” refers to a subclass of a multifactorial disease population consisting of members whose clinical presentation of the disease is more similar to each other than to the clinical presentation of the disease in members belonging to another homogeneic subclass of the same multifactorial disease.

To identify homogeneic subclasses of a given multifactorial disease, physiological profiling of the patient population is performed. Differences in correlation patterns identified from physiological profiles can be used to assign patients to homogeneic subclasses such that members of each subclass have a physiological profile distinct from patients in another subclass. Once the homogeneic subclasses of a multifactorial disease have been identified, a new patient can be diagnosed as belonging to a particular homogeneic subclass by physiological profiling and comparison of the new patient's physiological profile with physiological profiles representative of the different homogeneic subclasses. The ability to diagnose patients as belonging to particular homogeneic subclasses of a disease is useful for determining optimal therapeutic regimens. This is because different therapeutic methods can vary in effectiveness between subclasses. The ability to diagnose a patient as belonging to a particular homogeneic subclasses is also useful for determining prognosis, as particular homogeneic subclasses may have better survival or other clinical outcomes compared to other homogeneic subclasses.

In another embodiment, physiological profiling can be used to determine risk factors associated with developing a particular disease condition. For example, the physiological profiles of patients predisposed to hypertension can be compared to the physiological profiles of those not predisposed to hypertension. Correlation patterns associated with various degrees of predispositions can be identified, and the corresponding physiological profiles representative of various risk groups can be used to determine the risk group to which a new patient belongs. This is done by comparing the new patient's physiological profile with those profiles representing various risk groups.

In embodiments in which the physiological processes of one organism are compared to representative physiological profiles in order to, for example, predict outcome of a therapy (drug, surgical, biopharmaceutical), determine a prognosis once the disease is identified, or determine an initial prediction of predisposition (actuarial assessment of a person's health), a modified method of generating a physiological profile is used. In the case of an individual organism, the profile will be produced by assessing the relative distance of the physiological determinant value is from the mean population value of the same physiological determinant. This distance will then be used in a manner similar to the correlation value. The overall pattern of the profile then is analogous to the correlation matrix. Prognosis, diagnosis and predisposition can then be determined empirically by the similarity or difference in the individual's profile versus other patients' known outcomes with similar profiles or the population average. This predictive nature of the profile can be used for various organisms, for example, humans.

Physiological profiling can be used in combination with genetic linkage analysis to identify loci associated with different clinical presentations, i.e. symptoms or manifestations, of a multifactorial disease. Populations of patients having a multifactorial disease condition typically exhibit heterogeneous clinical presentations. Physiological profiling allows the heterogeneous patient population to be partitioned into more homogeneous subclasses. Genetic linkage analysis can identify chromosomal regions that are associated with particular phenotypic presentations. Combining physiological profiling with genetic linkage analysis data allows for identification of multiple genetic loci that may give rise to similar clinical presentations.

In another embodiment, physiological profiling can be used as a comprehensive approach to characterizing the influences of particular genomic regions on the relationships among pathways within complex physiological processes. Genetic linkage analysis alone reveals the direct influences of genes on the mechanisms measured by the mapped phenotypes. The influences of genes on mechanisms measured by the mapped phenotypes represent first order linkage. Physiological profiling allows for identification mechanistic relationships among pathways associated with complex physiological processes. When genetic linkage analysis is combined with physiological profiling, the effects of genotype on relationships among pathways within complex physiological process can be determined. Thus, combining genetic linkage analysis with physiological profiling provides a means to relate genetic information with functional pathways.

Physiological profiling also can be combined with expression profiling, either alone or in combination with genetic linkage analysis, to perform functional genomics. Expression profiling, as described, for example in U.S. Pat. Nos. 6,251,601; 5,800,992; and 5,445,934 can be used to identify genes that are expressed under particular conditions. Genetic linkage analysis identifies locations of the genome that are associated with particular phenotypic determinants. Physiological profiling identifies relationships among phenotypic determinants. Knowledge of the expression profiles of individual genes, their locations on chromosomes, and their effects on relationships among functional pathways within complex physiological processes can provide profound insights into the biology of organisms.

The invention will be further described in the following examples, which do not limit the scope of the invention described in the claims.

### EXAMPLES

### Example 1

### Animals Used in a Study on the Genetic Basis of Hypertension

F2 progeny rats derived from an intercross of an inbred hypertensive rat and a normotensive rat were used. The inbred hypertensive rat was a Dahl salt sensitive rat (SS/JrHsdMcw), and the inbred normotensive rat was a Brown Norway rat (BN/SsNHsdMcw). Two hundred and twelve F2 rats (113 males and 99 females) were extensively phenotyped for 239 mechanistically relevant cardiovascular, neuroendocrine, and renal phenotypes, including a number of cardiovascular stressors, both dietary and pharmacological, as described in Examples 2, 3, 4, and 5.

### Example 2

### Phenotyping Protocol for Conscious Animals at High and Low Salt Intakes

Rats were maintained on a high salt diet (8% salt) from the age of 9 to 13 weeks. During the fourth week of the high salt diet, arterial pressures of un-anesthetized rats were measured for three hours each day for three days. All blood pressure (BP) measurements were made with the animals unrestrained in their home cages as described previously (Cowley Jr. et al. (2000) Physiological Genomics 2:107-115). Implanted arterial catheters were used in determining arterial pressures. Data were collected at a rate of 100 Hz and reduced to one-minute averages; data for time series analysis were reduced to one-second averages. At the end of the third high salt day, animals were salt-depleted and placed on a low salt diet. One and a half days following furosemide-induced salt depletion and switching to a low salt diet, arterial pressure responses were determined. The day-night light cycle for all rats ran from 2:00 AM (lights on) to 2:00 PM (lights off) throughout the study.

Blood pressure data for high salt day 1 (BP1) consisted of baseline measurements of heart rate and systolic, diastolic, and mean arterial pressures measured from 9:00 AM to noon.

Blood pressure data for high salt day 2 (BP2) consisted of measurements of heart rate and systolic, diastolic, and mean arterial pressures obtained for the inactive (lights on) and active (lights off) phase. Data for the inactive phase (baseline data) were obtained from 9:00 AM to noon as was done for high salt day 1. Data for the active phase were obtained from 2:00 PM-6:00 PM. All blood pressure data on this day were collected for time-series analysis. A 24-hour urine collection was started in which urine volume as well as sodium, potassium, protein, and creatinine levels were determined.

Blood pressure data for high salt day 3 (BP3) consisted of baseline measurements of heart rate and systolic, diastolic, and mean arterial pressures measured from 9:00 AM to noon. Following the baseline measurements, a blood sample (500 μL) was drawn for determination of plasma renin activity and creatinine, plasma protein, and hematocrit levels. Following the blood draw, an injection of furosemide (10 mg/kg) was given intraperitoneally (ip) to salt deplete the animals. Following the furosemide administration, the animals were switched to a low salt diet (0.4% salt).

Blood pressure data for salt-depleted-day 4 (BP4) consisted of measurements of heart rate and systolic, diastolic, and mean arterial pressures measured from 9:00 AM to noon in the salt depleted state. These measurements were followed by a stress test. The stress test consisted of delivering two alerting stimuli five minutes apart; each alerting stimulus was 2 milliamps for 0.3 seconds. The change in mean arterial pressure, the time to peak, and the time to 90% recovery in response to the stress test were determined.

Blood pressure data for salt depleted-day 5 (BP5) consisted of measurements of heart rate and systolic, diastolic, and mean arterial pressures determined from 9:00 AM to noon in the salt depleted state. Following the recording period, a 1.0 mL blood sample was ₁aken for determination of plasma renin activity; white blood cell count; and triglycerides, total cholesterol, HDL, creatinine, and hematocrit levels.

### Example 3

### Phenotyping Protocol for Renal and Peripheral Vascular Reactivity in Anesthetized Animals

Rats were anesthetized with 30 mg/kg of ketamine and with 50 mg/kg of Inactin administered intraperitoneally. Catheters were implanted in the femoral artery and vein, and an electromagnetic flow probe was placed on the left renal artery via a midline incision. An intravenous (iv) infusion (50 μL/min) of isotonic saline containing 1% bovine serum albumin was performed to replaced fluid loss. After a 45-minute equilibration period, control values of arterial blood pressure and renal blood flow (RBF) were measured for 15 minutes. Next, animals were given iv infusions of angiotensin II (20, 100, 200 ng/kg/min) and norepinephrine (0.5, 1, 3 μg/kg/min) for 5 minutes after which renal and peripheral vascular responses were determined. Following recovery of pressure to baseline values, animals were given two successive doses of acetylcholine (ACh) (0.1 and 0.2 μg/kg/min as bolus doses) after which renal vascular and systemic arterial responses were measured. To determine the contribution of nitric oxide to basal renal vascular tone, 5 mg/kg of nitro-L-arginine methyl ester (L-NAME) was administered as an iv bolus, and then renal blood flow and renal resistance were determined. After 10 minutes of equilibration, the degree of blockade of the synthesis of nitric oxide produced by L-NAME was determined by administration of a repeat infusion of the same two doses of Ach, and renal blood flow and renal resistance were examined.

### Example 4

### Collection of Tissue Samples for Morphometric Measurements and Histology

To assess the degree of cardiac and renal hypertrophy, heart and kidneys were removed, stripped of surrounding tissue, and weighed using a digital top loading Sartorius balance. For histological analysis, the right kidney was fixed by immersion in 10% buffered formalin, embedded in paraffin, and the prepared sections were stained with hematoxylin and eosin as well as Periodic acid Schiff (PAS). These sections were evaluated for mean glomerular diameter and the degree of focal glomerulosclerosis. The degree of focal glomerulosclerosis was used as an index of glomerular injury.

### Example 5

### Development of a Genetic Linkage Map Determinant Phenotypes of Blood Pressure

To obtain a comprehensive picture of the genomic regions that are linked to blood pressure determinants, a genetic linkage map of measured and derived determinant phenotypes obtained as described in Examples 2, 3, and 4 was generated. These phenotypes represented critical elements of neuroendocrine, vascular, and renal functions. Table 1 below summarizes the physiological determinants used this study.

LOD threshold (Thrd) for Parametric analysis is 2.8 or 2.5 (in a few specific cases).
LOD threshold (Thrd) for Non_parametric analysis is 3.5.
One phenotype can be mapped on different chromosomes.
Group g1 through g18 and phen-group refer to ordering and grouping of phenotypes

| | Phen. | Phen. | | | | | LOD | |
|---|---|---|---|---|---|---|---|---|
| No. | group | grp. # | Phen. name | Phen. Description | Chr# | Thrd | Peak | Marker |
| 1 | RVR | g1 | TR_D_ANG1_M_CTRL_RVR_LN | Delta renal vascular resistance | 3 | 2.5 | 2.788 | D3Mgh23 |
| | ANG | | | from Angll dose 1 minus control | | | | |
| | | | | renal vascular resistance | | | | |
| 2 | RVR | g1 | TR_D_ANG2_M_CTRL_RVR_LN | Delta renal vascular resistance | 6 | 2.8 | 3.224 | D6Mit8 |
| | ANG | | | from Angll dose 2 minus control | | | | |
| | | | | renal vascular resistance | | | | |
| 3 | RVR | g1 | TR_D_ANG3_M_CTRL_RVR_LN | Delta renal vascular resistance | 5 | 2.5 | 2.798 | D5Mgh8 |
| | ANG | | | from Angll dose 3 minus control | | | | |
| | | | | renal vascular resistance | | | | |
| 4 | RVR NE | g2 | TR_ARVA_LS_PRE_NE_RVR_MEAN_LN | Control renal vascular resistance | 15 | 2.5 | 2.562 | D15Mgh11 |
| | | | | after Angll but before Norepi, | | | | |
| | | | | calculated by dividing RBF/g kwt | | | | |
| | | | | by MAP | | | | |
| 5 | RVR NE | g2 | TR_ARVA_LS_NE_D1_RVR_MEAN_LN | Norepinephrine dose 1 (0.5 ug/ | 1 | 2.5 | 2.535 | D1Mgh3 |
| | | | | kg/min), Renal vascular | | | | |
| | | | | resistance, calculated by dividing | | | | |
| | | | | RBF/g kwt by MAP | | | | |
| 6 | RVR NE | g2 | TR_ARVA_NE_SLOPE_RVR_LN | Slope of the regression for each | 6 | 2.8 | 2.876 | D6Mgh11 |
| | | | | rat; log dose of NE vs the RVR | | | | |
| | | | | corresponding to that dose for | | | | |
| | | | | each of three doses | | | | |
| 7 | RVR NE | g2 | TR_D_NE1_M_CTRL_RVR_LN | Delta renal vascular resistance | 10 | 2.8 | 3.873 | D10Mgh11 |
| | | | | from Norepinephrine dose 1 | | | | |
| | | | | minus control renal vascular | | | | |
| | | | | resistance | | | | |
| 8 | RVR | g3 | DELA_ACH2_M_CTRL_RVR | Delta renal vascular resistance | 5 | 2.8 | 4.149 | D5Mgh23 |
| | ACH | | | from Acetylcholine dose 2 minus | | | | |
| | | | | pre-Ach control renal vascular | | | | |
| | | | | resistance | | | | |
| 9 | RVR | g3 | TR_D_ACH4_M_LNAME_RVR_LN | Delta renal vascular resistance | 17 | 2.8 | 2.881 | D17Rat59 |
| | ACH | | | from Acetylcholine dose 4 minus | | | | |
| | | | | L-NAME renal vascular resistance | | | | |
| 10 | RVR | g3 | ARVA_LS_AII_2_RBF_MEAN | Angiotensin II dose 2 | 6 | 2.5 | 2.634 | D6Mit8 |
| | ACH | | | (96 ng/kg/min), Renal blood flow | | | | |
| | | | | per gram kidney weight, | | | | |
| | | | | anesthetized rat, mean of last two | | | | |
| | | | | steady state minutes of period | | | | |
| 11 | RBF ANG | g4 | DELTA_ANG2_M_CTRL_RBF | Delta renal blood flow from Angll | 12 | 2.8 | 2.998 | D12Mgh9 |
| | | | | dose 2 minus control renal blood | | | | |
| | | | | flow | | | | |
| 12 | RBF ANG | g4 | DELTA_ANG2_M_CTRL_RBFK | Delta renal blood flow per gram | 6 | 2.5 | 2.529 | D6Mgh4 |
| | | | | kidney weight from Angll dose 2 | | | | |
| | | | | minus control renal blood flow per | | | | |
| | | | | gram kidney weight | | | | |
| 13 | RBF ANG | g4 | DELTA_ANG3_M_CTRL_RBF | Delta renal blood flow from Angll | 12 | 2.5 | 2.547 | D12Mgh8 |
| | | | | dose 3 minus control renal blood | | | | |
| | | | | flow | | | | |
| 14 | RBF ANG | g4 | DELTA_ANG3_M_CTRL_RBFK | Delta renal blood flow per gram | 19 | 2.8 | 2.818 | D19Mit10 |
| | | | | kidney weight from Angll dose 3 | | | | |
| | | | | minus control renal blood flow per | | | | |
| | | | | gram kidney weight | | | | |
| 15 | RBF NE | g5 | ARVA_LS_PRE_NE_RBF_MEAN | Control renal blood flow per gram | 15 | 2.8 | 3.071 | D15Mgh11 |
| | | | | kidney weight after Angll but | | | | |
| | | | | before Norepi, anesthetized rat, | | | | |
| | | | | mean of last two steady state | | | | |
| | | | | minutes of control period | | | | |
| 16 | RBF NE | g5 | ARVA_NE_SLOPE_RBFK | Slope of the regression for each | 9 | 2.5 | 2.61 | D9Rat31 |
| | | | | rat; log dose of NE vs the RBF/g | | | | |
| | | | | kwt corresponding to that dose for | | | | |
| | | | | each of three doses | | | | |
| 17 | RBF ACH | g6 | ARVA_LS_ACH_1_RBF_MEAN | Acetylcholine dose 1 (0.095 ug/kg/ | 19 | 2.5 | 2.737 | D19Mit14 |
| | | | | min), Renal blood flow per | | | | |
| | | | | gram kidney weight, anesthetized | | | | |
| | | | | rat, mean of last two steady state | | | | |
| | | | | minutes of period | | | | |
| 18 | RBF ACH | g6 | ARVA_LS_ACH_D2_RBF_MEAN | Acetylcholine dose 2 (0.19 ug/kg/ | 19 | 2.5 | 3.104 | D19Mit14 |
| | | | | min), Renal blood flow per | | | | |
| | | | | gram kidney weight, anesthetized | | | | |
| | | | | rat, mean of last two steady state | | | | |
| | | | | minutes of period | | | | |
| 19 | RBF ACH | g6 | ARVA_LS_ACH3_RBF_MEAN | Acetylcholine dose 3 (0.095 ug/kg/ | 15 | 2.5 | 2.59 | D15Mgh11 |
| | | | | min), Renal blood flow per | | | | |
| | | | | gram kidney weight, anesthetized | | | | |
| | | | | rat, mean of last two steady state | | | | |
| | | | | minutes of period | | | | |
| 20 | RBF ACH | g6 | DELTA_ACH1_M_CTRL_RBF | Delta renal blood flow from | 5 | 3.5 | 4.16275 | D5Mgh5 |
| | | | | Acetylcholine dose 1 minus pre- | | | | |
| | | | | Ach control renal blood flow | | | | |
| 21 | RBF ACH | g6 | DELTA_ACH1_M_CTRL_RBFK | Delta renal blood flow per gram | 5 | 3.5 | 4.36965 | D5Mgh5 |
| | | | | kidney weight from Acetylcholine | | | | |
| | | | | dose 1 minus pre-Ach control | | | | |
| | | | | renal blood flow per gram kidney | | | | |
| | | | | weight | | | | |
| 22 | RBF ACH | g6 | DELTA_ACH2_M_CTRL_RBF | Delta renal blood flow from | 5 | 2.8 | 3.296 | D5Mit4 |
| | | | | Acetylcholine dose 2 minus pre- | | | | |
| | | | | Ach control renal blood flow | | | | |
| 23 | RBF ACH | g6 | DELTA_ACH4_M_LNAME_RBF | Delta renal blood flow from | 3 | 2.8 | 2.814 | D3Rat116 |
| | | | | Acetylcholine dose 4 minus L- | | | | |
| | | | | NAME renal blood flow | | | | |
| 24 | RBF ACH | g6 | DELTA_ACH4_M_LNAME_RBFK | Delta renal blood flow per gram | 3 | 2.8 | 2.895 | D3Rat116 |
| | | | | kidney weight from Acetylcholine | | | | |
| | | | | dose 4 minus L-NAME renal blood | | | | |
| | | | | flow per gram kidney weight | | | | |
| 25 | EXCR | g7 | TR_RF_HS_URINE_VOL_SQT | 24 hour urine volume in ml, rat on | 4 | 2.8 | 2.834 | D4Mit27 |
| | | | | high salt diet | | | | |
| | EXCR | g7 | TR_RF_HS_URINE_VOL_SQT | 24 hour urine volume in ml, rat on | 8 | 2.8 | 3.871 | D8Mit16 |
| | | | | high salt diet | | | | |
| 26 | EXCR | g7 | RF_LS_URINE_NA | sodium concentration of urine, low | 1 | 2.8 | 4.643 | D1Mgh3 |
| | | | | salt diet following Lasix | | | | |
| 27 | EXCR | g7 | RF_LS_24HR_EXCR_NA | sodium excretion rate, low salt | 1 | 2.5 | 2.573 | D1Mgh3 |
| | | | | diet fillowing Lasix | | | | |
| 28 | EXCR | g7 | RF_HS_24HR_EXCR_K | Postassium excretion rate, high salt | 12 | 2.5 | 2.677 | D12Rat43 |
| | | | | diet | | | | |
| 29 | EXCR | g7 | TR_RF_HS_URINE_K_LN | potassium concentration of urine, | 16 | 2.5 | 2.883 | D16Mit2 |
| | | | | high salt diet | | | | |
| 30 | EXCR | g7 | TR_RF_LS_24HR_EXCR_K_LN | Potassium excretion rate, low salt | 1 | 2.8 | 4.66 | D1Mit10 |
| | | | | diet following Lasix | | | | |
| 31 | EXCR | g7 | TR_RF_LS_URINE_K_LN | potassium concentration of urine, | 1 | 2.8 | 4.589 | D1Mit10 |
| | | | | low salt diet following Lasix | | | | |
| | EXCR | g7 | TR_RF_LS_URINE_K_LN | potassium concentration of urine, | 3 | 2.8 | 3.708 | D3Mgh6 |
| | | | | low salt diet following Lasix | | | | |
| | EXCR | g7 | TR_RF_LS_URINE_K_LN | potassium concentration of urine, | 4 | 2.8 | 2.988 | D4Mit2 |
| | | | | low salt diet following Lasix | | | | |
| 32 | KID | g8 | TR_RF_HS_24HR_URINE_CREAT_LN | creatinine concentration of urine, | 2 | 2.5 | 2.717 | D2Mgh14 |
| | FUNC | | | high salt diet | | | | |
| 33 | KID | g8 | TR_RF_HS_24HR_EXCR_PROTEIN_LN | protein excretion rate, high salt | 18 | 2.8 | 2.981 | D18Mgh9 |
| | FUNC | | | diet | | | | |
| 34 | KID | g8 | TR_RF_HS_EXCR_PROT_MG24HR_LN | protein excretion rate, high salt | 18 | 2.8 | 2.933 | D18Mgh7 |
| | FUNC | | | diet | | | | |
| 35 | KID | g8 | TR_RF_HS_24HR_URINE_PROTEIN_LN | protein concentration of urine, | 8 | 2.8 | 2.924 | D8Mit14 |
| | FUNC | | | high salt diet | | | | |
| 36 | KID | g8 | AP_RGHT_KIDNEY_WGHT | right kidney weight | 7 | 2.8 | 3.934 | D7Mit14 |
| | FUNC | | | | | | | |
| | KID | g8 | AP_RGHT_KIDNEY_WGHT | right kidney weight | 12 | 2.8 | 3.571 | D12Mit7 |
| | FUNC | | | | | | | |
| 37 | KID | g8 | AP_LFT_KIDNEY_WGHT | left kidney weight | 7 | 3.5 | 4.62096 | D7Mit14 |
| | FUNC | | | | | | | |
| 38 | BP | g9 | RAWBP_DAY1_MAP | mean blood pressure, High salt | 18 | 3.5 | 3.53873 | D18Rat57 |
| | | | | day 1, mean of 3 hour blood | | | | |
| | | | | pressure recording | | | | |
| 39 | BP | g9 | RAWBP_DAY2_DAP | Diastolic blood pressure, High | 18 | 2.8 | 2.829 | D18Rat57 |
| | | | | salt, day 2, mean of 3 hour blood | | | | |
| | | | | pressure recording | | | | |
| 40 | BP | g9 | RAWBP_DAY2_MAP | mean blood pressure, High salt, | 18 | 3.5 | 4.41065 | D18Rat57 |
| | | | | day 2, mean of 3 hour blood | | | | |
| | | | | pressure recording | | | | |
| 41 | BP | g9 | RAWBP_DAY3_DAP | Diastolic blood pressure, High | 13 | 2.5 | 2.583 | D13Mgh18 |
| | | | | salt, day 3, mean of 3 hour blood | | | | |
| | | | | pressure recording | | | | |
| 42 | BP | g9 | TR_BPX_HSBASALDIAMEAN_LN | Diastolic blood pressure, arterial | 18 | 2.5 | 2.655 | D18Rat57 |
| | | | | catheter implanted, high salt diet, | | | | |
| | | | | mean of best 2 out of 3 days, 3 | | | | |
| | | | | hours collection time per day, | | | | |
| | | | | basal state - lights on and rat | | | | |
| | | | | asleep, | | | | |
| 43 | BP | g9 | TR_BPX_LSBASALDIAMEAN_LN | Diastolic blood pressure, arterial | 14 | 2.5 | 2.74 | D14MiT7 |
| | | | | catheter implanted, low salt diet | | | | |
| | | | | following Lasix, 3 hours collection | | | | |
| | | | | time one day, basal state - lights | | | | |
| | | | | on and rat asleep, | | | | |
| 44 | BP | g9 | TR_BPX_HSACTIVEDIAMEAN_LN | Diastolic blood pressure, arterial | 18 | 2.5 | 2.791 | D18Rat57 |
| | | | | catheter implanted, high salt diet, | | | | |
| | | | | average of 3 hours collection time, | | | | |
| | | | | active state - lights off and rat | | | | |
| | | | | awake | | | | |
| 45 | BP | g9 | BPX_HSBASALMAPMEAN | Mean arterial blood pressure, | 18 | 3.5 | 3.88752 | D18Rat57 |
| | | | | arterial catheter implanted, high | | | | |
| | | | | salt diet, mean of best 2 out of 3 | | | | |
| | | | | days, 3 hours collection time per | | | | |
| | | | | day, basal state - lights on and rat | | | | |
| | | | | asleep, | | | | |
| 46 | BP | g9 | BPX_HSACTIVEMAPMEAN | Mean arterial pressure, arterial | 18 | 3.5 | 4.36484 | D18Rat57 |
| | | | | catheter implanted, high salt diet, | | | | |
| | | | | average of 3 hours collection time, | | | | |
| | | | | active state - lights off and rat | | | | |
| | | | | awake | | | | |
| 47 | BP | g9 | DELTA_WAKE_M_DAY2AM_SYSBP | systolic blood pressure, high salt | 2 | 3.5 | 3.66819 | D2Mgh29 |
| | | | | diet, p.m. active state value minus | | | | |
| | | | | a.m. basal state (mean of 3 hours | | | | |
| | | | | recording each) | | | | |
| | BP | g9 | DELTA_WAKE_M_DAY2AM_SYSBP | systolic blood pressure, high salt | 15 | 3.5 | 3.53715 | D15Mgh9 |
| | | | | diet, p.m. active state value minus | | | | |
| | | | | a.m. basal state (mean of 3 hours | | | | |
| | | | | recording each) | | | | |
| 48 | BP | g9 | DELTA_WAKE_M_DAY2AM_DIABP | diastolic blood pressure, high salt | 13 | 2.5 | 2.524 | D13Mit4 |
| | | | | diet, p.m. active state value minus | | | | |
| | | | | a.m. basal state (mean of 3 hours | | | | |
| | | | | recording each) | | | | |
| 49 | BP | g9 | DELTA_WAKE_M_DAY2AM_MAPBP | mean arterial blood pressure, high | 13 | 2.5 | 2.973 | D13Mit4 |
| | | | | salt diet, p.m. active state value | | | | |
| | | | | minus a.m. basal state (mean of 3 | | | | |
| | | | | hours recording each) | | | | |
| 50 | BP | g9 | DELTA_HS_M_LS_MAPBP | mean arterial pressure, high salt | 18 | 3.5 | 4.61609 | D18Rat57 |
| | | | | minus low salt, basal state - lights | | | | |
| | | | | on and rat asleep | | | | |
| 51 | BP | g9 | DELTA_HS_M_LS_SYSBP | Systolic blood pressure, high salt | 8 | 2.8 | 3.398 | D8Mit4 |
| | | | | minus low salt, basal state - lights | | | | |
| | | | | on and rat asleep | | | | |
| | BP | g9 | DELTA_HS_M_LS_SYSBP | Systolic blood pressure, high salt | 18 | 2.8 | 3.643 | D18Mit3 |
| | | | | minus low salt, basal state - lights | | | | |
| | | | | on and rat asleep | | | | |
| 52 | BP SD | g10 | DELTA_HS_M_LS_MAPSD | mean arterial pressure standard | 3 | 2.8 | 2.848 | D3Mit4 |
| | | | | deviation, high salt minus low | | | | |
| | | | | salt, basal state - lights on and rat | | | | |
| | | | | asleep | | | | |
| | BP SD | g10 | DELTA_HS_M_LS_MAPSD | mean arterial pressure standard | 7 | 2.8 | 2.825 | D7Rat135 |
| | | | | deviation, high salt minus low | | | | |
| | | | | salt, basal state - lights on and rat | | | | |
| | | | | asleep | | | | |
| 53 | BP SD | g10 | TR_BPX_HSACTIVEMAPSD_LN | Mean arterial pressure standard | 1 | 2.8 | 3.836 | D1Mit2 |
| | | | | deviation, arterial catheter | | | | |
| | | | | implanted, high salt diet, average | | | | |
| | | | | of 3 hours collection time, active | | | | |
| | | | | state - lights off and rat awake | | | | |
| 54 | BP SD | g10 | TR_BPX_LSBASALDIASD_SQT | Diastolic blood pressure standard | 13 | 2.8 | 2.98 | D13Mgh18 |
| | | | | deviation, arterial catheter | | | | |
| | | | | implanted, low salt diet following | | | | |
| | | | | Lasix, 3 hours collection time one | | | | |
| | | | | day, basal state - lights on and rat | | | | |
| | | | | asleep, | | | | |
| 55 | BP SD | g10 | TR_BPX_LSBASALMAPSD_LN | Mean arterial pressure standard | 3 | 2.5 | 2.728 | D3Mit4 |
| | | | | deviation, arterial catheter | | | | |
| | | | | implanted, low salt diet following | | | | |
| | | | | Lasix, 3 hours collection time one | | | | |
| | | | | day, basal state - lights on and rat | | | | |
| | | | | asleep, | | | | |
| 56 | BP SD | g10 | TR_D_WAKE_M_DAY2AM_MAPSD_SQT | mean arterial blood pressure | 8 | 2.5 | 2.574 | D8Mgh4 |
| | | | | standard deviation, high salt diet, | | | | |
| | | | | p.m. active state value minus a.m. | | | | |
| | | | | basal state (mean of 3 hours | | | | |
| | | | | recording each) | | | | |
| 57 | BP SD | g10 | TR_RAWBP_DAY1_SAPSD_LN | Systolic blood pressure standard | 2 | 2.5 | 2.687 | D2Mgh16 |
| | | | | deviation, High salt, day 1, mean | | | | |
| | | | | of 3 hour blood pressure | | | | |
| | | | | recording | | | | |
| 58 | BP SD | g10 | TR_RAWBP_DAY3_DAPSD_LN | Diastolic blood pressure standard | 2 | 2.8 | 3.665 | D2Mgh12 |
| | | | | deviation, High salt, day 3, mean | | | | |
| | | | | of 3 hour blood pressure | | | | |
| | | | | recording | | | | |
| 59 | BP SD | g10 | TR_RAWBP_DAY3_MAPSD_LN | mean blood pressure standard | 2 | 2.8 | 4.377 | D2Mgh12 |
| | | | | deviation, High salt, day 3, mean | | | | |
| | | | | of 3 hour blood pressure | | | | |
| | | | | recording | | | | |
| 60 | BP SD | g10 | BPX_HSACTIVEDIASD | Diastolic blood pressure standard | 1 | 3.5 | 3.60022 | D1Mit3 |
| | | | | deviation, arterial catheter | | | | |
| | | | | implanted, high salt diet, average | | | | |
| | | | | of 3 hours collection time, active | | | | |
| | | | | state - lights off and rat awake | | | | |
| 61 | BP SD | g10 | BPX_HSBASALMAPSD | Mean arterial pressure standard | 2 | 3.5 | 3.61192 | D2Mgh12 |
| | | | | deviation, arterial catheter | | | | |
| | | | | implanted, high salt diet, mean of | | | | |
| | | | | best 2 out of 3 days, 3 hours | | | | |
| | | | | collection time per day, basal | | | | |
| | | | | state - lights on and rat asleep, | | | | |
| 62 | BP SD | g10 | RAWBP_DAY2_DAPSD | Diastolic blood pressure standard | 18 | 3.5 | 3.59112 | D18Rat57 |
| | | | | deviation, High salt, day 2, mean | | | | |
| | | | | of 3 hour blood pressure | | | | |
| | | | | recording | | | | |
| 63 | BP SD | g10 | RAWBP_DAY2_MAPSD | mean blood pressure standard | 18 | 3.5 | 3.97196 | D.18Rat57 |
| | | | | deviation, High salt, day 2, mean | | | | |
| | | | | of 3 hour blood pressure | | | | |
| | | | | recording | | | | |
| 64 | BP SD | g10 | RAWBP_DAY3_SAPSD | Systolic blood pressure standard | 1 | 3.5 | 3.71601 | D1Mit2 |
| | | | | deviation, High salt, day 3, mean | | | | |
| | | | | of 3 hour blood pressure | | | | |
| | | | | recording | | | | |
| 65 | BP | g11 | BPTSM_TAM_ALPHA1 | Tuesday a.m. Linear term 0th | 7 | 2.8 | 4.043 | D7Mit10 |
| | T-SERIES | | | order parameter (mechanistic | | | | |
| | | | | model) | | | | |
| 66 | BP | g11 | BPTSM_TAM_ALPHA2 | Tuesday a.m. Linear term 1st | 7 | 2.5 | 4.773 | D7Mit10 |
| | T-SERIES | | | order parameter (mechanistic | | | | |
| | | | | model) | | | | |
| 67 | BP | g11 | BPTSM_TAM_ALPHA3 | Tuesday a.m. Linear term 2nd | 13 | 2.5 | 2.55 | D13Mit4 |
| | T-SERIES | | | order parameter (mechanistic | | | | |
| | | | | model) | | | | |
| 68 | BP | g11 | BPTSM_TAM_U | Tuesday a.m. Exponential set | 14 | 2.5 | 2.556 | D14Rat1 |
| | T-SERIES | | | point, baro-receptor response | | | | |
| | | | | (mechanistic model) | | | | |
| 69 | BP | g11 | BPTSM_TPM_ALPHA1 | Tuesday p.m. Linear term 0th | 2 | 2.8 | 3.476 | D2Mgh1 |
| | T-SERIES | | | order parameter (mechanistic | | | | |
| | | | | model) | | | | |
| 70 | BP | g11 | BPTSM_TPM_ALPHA2 | Tuesday p.m. Linear term 1st | 5 | 2.8 | 3.413 | D5Rat178 |
| | T-SERIES | | | order parameter (mechanistic | | | | |
| | | | | model) | | | | |
| | BP | g11 | BPTSM_TPM_ALPHA2 | Tuesday p.m. Linear term 1st | 18 | 2.8 | 3.807 | D18Mgh3 |
| | T-SERIES | | | order parameter (mechanistic | | | | |
| | | | | model) | | | | |
| | BP | g11 | BPTSM_TPM_ALPHA2 | Tuesday p.m. Linear term 1st | 18 | 2.8 | 3.201 | D18Mgh9 |
| | T-SERIES | | | order parameter (mechanistic | | | | |
| | | | | model) | | | | |
| 71 | BP | g11 | BPTSM_TPM_SD | Tuesday p.m. standard deviation | 3 | 2.5 | 2.639 | D3Rat27 |
| | T-SERIES | | | of blood pressure | | | | |
| 72 | BP | g11 | BPTSM_TPM_U | Tuesday p.m. Exponential set | 18 | 2.5 | 2.542 | D18Rat57 |
| | T-SERIES | | | point, baro-receptor response | | | | |
| | | | | (mechanistic model) | | | | |
| 73 | BP | g11 | BPTSM_WAM_ALPHA1 | Wednesday a.m. Linear term 0th | 4 | 2.5 | 2.515 | D4Mgh1 |
| | T-SERIES | | | order parameter (mechanistic | | | | |
| | | | | model) | | | | |
| 74 | BP | g11 | BPTSM_WAM_ALPHA2 | Wednesday a.m. Linear term 1st | 18 | 2.8 | 3.022 | D18Mgh7 |
| | T-SERIES | | | order parameter (mechanistic | | | | |
| | | | | model) | | | | |
| 75 | BP | g11 | BPTSM_WAM_ALPHA3 | Wednesday a.m. Linear term 2nd | 2 | 2.5 | 2.597 | D2Mit4 |
| | T-SERIES | | | order parameter (mechanistic | | | | |
| | | | | model) | | | | |
| 76 | BP | g11 | BPTSM_WAM_XBAR | Wednesday a.m. Set point for | 18 | 2.8 | 3.053 | D18Rat57 |
| | T-SERIES | | | baro-receptor response | | | | |
| | | | | (mechanistic model) | | | | |
| 77 | BP | g11 | BPTSM_TAM_ADA | Tuesday a.m. Exponential scaling | 13 | 3.5 | 3.72886 | D13Mit4 |
| | T-SERIES | | | factor, baro-receptor response | | | | |
| | | | | (mechanistic model) | | | | |
| 78 | BP | g11 | BPTSM_TPM_ALPHA3 | Tuesday p.m. Linear term 2nd | 6 | 3.5 | 3.66407 | D6Rat163 |
| | T-SERIES | | | order parameter (mechanistic | | | | |
| | | | | model) | | | | |
| 79 | BP | g11 | BPTSM_WAM_D | Wednesday a.m. fractal | 2 | 3.5 | 3.62454 | D2Mgh26 |
| | T-SERIES | | | parameter (fARIMA model) | | | | |
| 80 | BP | g11 | TR_BPTSM_TAM_MEAN_LN | Tuesday a.m. mean blood | 18 | 2.8 | 3.269 | D18Rat57 |
| | T-SERIES | | | pressure | | | | |
| 81 | BP | g11 | TR_BPTSM_TAM_SD_LN | Tuesday a.m. standard deviation | 4 | 2.8 | 2.979 | D4Mgh12 |
| | T-SERIES | | | of blood pressure | | | | |
| 82 | BP DRUG | g12 | ARVA_LS_CNTRL_MAP_MEAN | Control mean arterial pressure, | 12 | 2.8 | 2.973 | D12Mgh5 |
| | | | | anesthetized rat, mean of last two | | | | |
| | | | | steady state minutes of control | | | | |
| | | | | period | | | | |
| 83 | BP DRUG | g12 | ARVA_AII_SLOPE_BP | Slope of the regression for each | 13 | 2.5 | 2.531 | D13Mgh18 |
| | | | | rat; log dose of All vs the BP | | | | |
| | | | | corresponding to that dose for | | | | |
| | | | | each of three doses | | | | |
| 84 | BP DRUG | g12 | TR_ARVA_NE_SLOPE_BP_SQT | Slope of the regression for each | | | | |
| | | | | rat; log dose of NE vs the BP | | | | |
| | | | | corresponding to that dose for | | | | |
| | | | | each of three doses | | | | |
| 85 | BP DRUG | g12 | ARVA_LS_All_1_MAP_MEAN | Angiotensin II dose 1 (20 ng/kg/ | | | | |
| | | | | min), Mean arterial | | | | |
| | | | | pressure, anesthetized rat, mean | | | | |
| | | | | of last two steady state minutes of | | | | |
| | | | | period | | | | |
| 86 | BP DRUG | g12 | ARVA_LS_AII_2_MAP_MEAN | Angiotensin II dose 2 | | | | |
| | | | | (96 ng/kg/min), Mean arterial | | | | |
| | | | | pressure, anesthetized rat, mean | | | | |
| | | | | of last two steady state minutes of | | | | |
| | | | | period | | | | |
| 87 | BP DRUG | g12 | ARVA_LS_AII_3_MAP_MEAN | Angiotensin II dose 2 | | | | |
| | | | | (192 ng/kg/min), Mean arterial | | | | |
| | | | | pressure, anesthetized rat, mean | | | | |
| | | | | of last two steady state minutes of | | | | |
| | | | | period | | | | |
| 88 | BP DRUG | g12 | ARVA_LS_PRE_NE_MAP_MEAN | Control mean arterial pressure | 1 | 2.8 | 3.127 | D1Mit18 |
| | | | | after AngII but before Norepi, | | | | |
| | | | | anesthetized rat, mean of last two | | | | |
| | | | | steady state minutes of control | | | | |
| | | | | period | | | | |
| 89 | BP DRUG | g12 | ARVA_LS_NE_1_MAP_MEAN | Norepinephrine dose 1 (0.5 ug/ | | | | |
| | | | | kg/min), Mean arterial | | | | |
| | | | | pressure, anesthetized rat, mean | | | | |
| | | | | of last two steady state minutes of | | | | |
| | | | | period | | | | |
| 90 | BP DRUG | g12 | ARVA_LS_NE_2_MAP_MEAN | Norepinephrine dose 2 (1.04 ug/kg/ | 17 | 2.5 | 2.593 | D17Rat32 |
| | | | | min), Mean arterial | | | | |
| | | | | pressure, anesthetized rat, mean | | | | |
| | | | | of last two steady state minutes of | | | | |
| | | | | period | | | | |
| 91 | BP DRUG | g12 | ARVA_LS_NE_3_MAP_MEAN | Norepinephrine dose 2 (2.96 ug/ | | | | |
| | | | | kg/min), Mean arterial | | | | |
| | | | | pressure, anesthetized rat, mean | | | | |
| | | | | of last two steady state minutes of | | | | |
| | | | | period | | | | |
| 92 | BP DRUG | g12 | ARVA_LS_PRE_ACH_1_MAP_MEAN | Control mean arterial pressure | | | | |
| | | | | after Norepi but before | | | | |
| | | | | Acetylcholine, anesthetized rat, | | | | |
| | | | | mean of last two steady state | | | | |
| | | | | minutes of control period | | | | |
| 93 | BP DRUG | g12 | ARVA_LS_ACH_1_MAP_MEAN | Acetylcholine dose 1 (0.095 ug/ | 10 | 2.5 | 2.504 | D10Mgh14 |
| | | | | kg/min), Mean arterial | | | | |
| | | | | pressure, anesthetized rat, mean | | | | |
| | | | | of last two steady state minutes of | | | | |
| | | | | period | | | | |
| 94 | BP DRUG | g12 | ARVA_LS_ACH_D2_MAP_MEAN | Acetylcholine dose 2 (0.19 ug/ | 10 | 2.5 | 2.792 | D10Mgh23 |
| | | | | kg/min), Mean arterial | | | | |
| | | | | pressure, anesthetized rat, mean | | | | |
| | | | | of last two steady state minutes of | | | | |
| | | | | period | | | | |
| 95 | BP DRUG | g12 | ARVA_LS ACH3_MAP_MEAN | Acetylcholine dose 3 (0.095 ug/ | | | | |
| | | | | kg/min), Mean arterial | | | | |
| | | | | pressure, anesthetized rat, mean | | | | |
| | | | | of last two steady state minutes of | | | | |
| | | | | period | | | | |
| 96 | BP DRUG | g12 | ARVA_LS_ACH_D4_MAP_MEAN | Acetylcholine dose 4 (0.19 ug/ | 12 | 2.8 | 3.041 | D12Mit7 |
| | | | | kg/min), Mean arterial | | | | |
| | | | | pressure, anesthetized rat, mean | | | | |
| | | | | of last two steady state minutes of | | | | |
| | | | | period | | | | |
| 97 | BP DRUG | g12 | DELTA_ACH2_M_CTRL_BP | Delta mean arterial pressure from | | | | |
| | | | | acetylcholine dose 2 minus pre- | | | | |
| | | | | Ach control mean arterial | | | | |
| | | | | pressure | | | | |
| 98 | BP DRUG | g12 | DELTA_ANG1_M_CTRL_BP | Delta mean arterial pressure from | 7 | 2.8 | 2.851 | D7Mit10 |
| | | | | AngII dose 1 minus control mean | | | | |
| | | | | arterial pressure | | | | |
| 99 | BP DRUG | g12 | DELTA_ANG3_M_CTRL_BP | Delta mean arterial pressure from | 13 | 2.8 | 2.944 | D13Mgh4 |
| | | | | AngII dose 3 minus control mean | | | | |
| | | | | arterial pressure | | | | |
| 100 | BP DRUG | g12 | DELTA_NE3_M_CTRL_BP | Delta mean arterial pressure from | 3 | 2.8 | 3.129 | D3Mgh18 |
| | | | | Norepinephrine dose 3 minus | | | | |
| | | | | control mean arterial pressure | | | | |
| 101 | BP DRUG | g12 | DELTA_ACH3_M_LNAME_BP | Delta mean arterial pressure from | 6 | 3.5 | 3.52475 | D6Rat163 |
| | | | | acetylcholine dose 3 minus L- | | | | |
| | | | | NAME mean arterial pressure | | | | |
| 102 | BP DRUG | g12 | TR_D_LNAME_M_ACH2_BP_LN | Delta mean arterial pressure from | 2 | 2.8 | 3.035 | D2Mgh15 |
| | | | | L-NAME minus Acetylcholine | | | | |
| | | | | dose 2 mean arterial pressure | | | | |
| 103 | BP DRUG | g12 | DELTA_ACH1_M_CTRL_BP | Delta mean arterial pressure from | 6 | 2.8 | 3.291 | D6Rat78 |
| | | | | acetylcholine dose 1 minus pre- | | | | |
| | | | | Ach control mean arterial | | | | |
| | | | | pressure | | | | |
| 104 | NEURO- | g13 | BW_LS_PLASMA_RENIN | Plasma renin, low salt diet | 1 | 2.8 | 4.467 | D1Mit10 |
| | ENDO- | | | following Lasix | | | | |
| | CRINE | | | | | | | |
| | NEURO- | g13 | BW_LS_PLASMA_RENIN | Plasma renin, low salt diet | 4 | 2.8 | 2.822 | D4Mgh7 |
| | ENDO- | | | following Lasix | | | | |
| | CRINE | | | | | | | |
| 105 | NEURO- | g13 | TR_BW_RENIN_LS_MINUS_HS_SQT | Change in plasma renin; low salt | 4 | 2.8 | 3.045 | D4Mgh7 |
| | ENDO- | | | minus high salt value | | | | |
| | CRINE | | | | | | | |
| 106 | HR | g14 | RAWBP_DAY1_HRTRT | heart rate, High salt, day 1, mean | 2 | 2.8 | 3.165 | D2Rat64 |
| | | | | of 3 hour blood pressure | | | | |
| | | | | recording | | | | |
| | HR | g14 | RAWBP_DAY1_HRTRT | heart rate, High salt, day 1, mean | 20 | 2.8 | 3.163 | RS19b |
| | | | | of 3 hour blood pressure | | | | |
| | | | | recording | | | | |
| 107 | HR | g14 | BPX_HSACTIVEHRMEAN | Heart rate, arterial catheter | 10 | 2.5 | 2.575 | D10Mgh11 |
| | | | | implanted, high salt diet, average | | | | |
| | | | | of 3 hours collection time, active | | | | |
| | | | | state - lights off and rat awake | | | | |
| 108 | HR | g14 | DELTA_HS_M_LS_HR | heart rate, high salt minus low | 5 | 2.8 | 3.006 | D5Rat178 |
| | | | | salt, basal state - lights on and rat | | | | |
| | | | | asleep | | | | |
| 109 | HR | g14 | DELTA_WAKE_M_DAY2AM_HR | heart rate, high salt diet, p.m. | 1 | 2.8 | 2.973 | D1Mgh7 |
| | | | | active state value minus a.m. | | | | |
| | | | | basal state (mean of 3 hours | | | | |
| | | | | recording each) | | | | |
| 110 | HR SD | g15 | TR_RAWBP_DAY1_HRTRTSD_LN | heart rate standard deviation, | 2 | 2.8 | 3.373 | D2Mit15 |
| | | | | High salt, day 1, mean of 3 hour | | | | |
| | | | | blood pressure recording | | | | |
| 111 | HR SD | g15 | RAWBP_DAY2_HRTRTSD | heart rate standard deviation, | 12 | 3.5 | 3.56232 | D12Rat43 |
| | | | | High salt, day 2, mean of 3 hour | | | | |
| | | | | blood pressure recording | | | | |
| 112 | HR SD | g15 | RAWBP_DAY3_HRTRTSD | heart rate standard deviation, | 4 | 3.5 | 3.54628 | D4Mit1 |
| | | | | High salt, day 3, mean of 3 hour | | | | |
| | | | | blood pressure recording | | | | |
| 113 | HR SD | g15 | TR_D_WAKE_M_DAY2AM_HRSD_LN | heart rate standard deviation, high | 17 | 2.8 | 4.094 | D17Rat9 |
| | | | | salt diet, p.m. active state value | | | | |
| | | | | minus a.m. basal state (mean of 3 | | | | |
| | | | | hours recording each) | | | | |
| 114 | LIPIDS | g16 | BW_HDL_VALUE | Plasma HDL value, low salt diet | 18 | 2.8 | 3.826 | D18Mit8 |
| | | | | following Lasix | | | | |
| | LIPIDS | g16 | BW_HDL_VALUE | Plasma HDL value, low salt diet | 18 | 2.8 | 3.995 | D18Mgh7 |
| | | | | following Lasix | | | | |
| 115 | LIPIDS | g16 | BW_LS_TRIGLYCERIDE | Plasma triglyceride, low salt diet | 1 | 2.5 | 2.691 | D1Mgh3 |
| | | | | following Lasix | | | | |
| 116 | LIPIDS | g16 | BW_TOTAL_CHOLESTEROL_VALUE | Plasma total cholesterol value, | 1 | 2.8 | 3.013 | D1Mgh7 |
| | | | | low salt diet following Lasix | | | | |
| | LIPIDS | g16 | BW_TOTAL_CHOLESTEROL_VALUE | Plasma total cholesterol value, | 5 | 2.8 | 3.065 | D5Mgh25 |
| | | | | low salt diet following Lasix | | | | |
| 117 | MORPHO | g17 | RAT_WGHT | Body weight in grams, prior to | 3 | 2.5 | 2.584 | D3Rat27 |
| | | | | anesthesia | | | | |
| 118 | MORPHO | g17 | RAT_WGHTCS | Body weight in grams following | 8 | 2.5 | 2.545 | D8Mit13 |
| | | | | catheter implantation and jacket | | | | |
| | | | | and spring fitting | | | | |
| 119 | MORPHO | g17 | RF_DELTA_WGHT | Change in body weight between | 7 | 2.8 | 3.348 | D7Rat135 |
| | | | | day of catheter surgery and day of | | | | |
| | | | | Lasix injection | | | | |
| 120 | MORPHO | g17 | RF_WGHT | Body weight of rat prior to Lasix | 14 | 2.8 | 3.028 | D14Mit7 |
| | | | | injection | | | | |
| 121 | MORPHO | g17 | TR_AP_LFT_VENTRICLE_WGHT_LN | left ventricle weight in grams | 14 | 2.8 | 2.971 | D14Mit7 |
| 122 | MORPHO | g17 | AP_HEART_WGHT | heart weight in grams | 2 | 3.5 | 3.50746 | D2Mgh12 |
| | MORPHO | g17 | AP_HEART_WGHT | heart weight in grams | 8 | 3.5 | 3.77443 | D8Mit13 |
| | MORPHO | g17 | AP_HEART_WGHT | heart weight in grams | 10 | 3.5 | 3.84606 | D10Mgh23 |
| 123 | MORPHO | g17 | ARVA_WGHTCS | Body weight prior to anesthesia | 3 | 2.8 | 3.503 | D3Mgh6 |
| | | | | for acute protocol | | | | |
| | MORPHO | g17 | ARVA_WGHTCS | Body weight prior to anesthesia | 18 | 2.8 | 2.963 | D18Mit8 |
| | | | | for acute protocol | | | | |
| 124 | MISC | g18 | AP_AORTA_LESION_NUMBER | number of aortic lesions found in | 11 | 3.5 | 3.64538 | D11Mgh3 |
| | | | | specimen analyzed | | | | |
| 125 | MISC | g18 | TR_BW_WBC_LN | White blood cell count | 12 | 2.8 | 3.162 | D12Mgh5 |

Determinant phenotypes were tested for normalcy as described in Cowley Jr. et al. (2000) Physiological Genomics 2:107-115. The 166 phenotypes that passed a test for being distributed as a normal random variable were analyzed via parametric methods in a genome scan using MAPMAKER/QTL (as described in J. P. Rapp (2000) Physiol. Rev. 80:135-172 and Hollenberg et al. (1978) Medicine 57:167-178). The remaining 73 phenotypes that did not fulfill the requirements for parametric analysis were analyzed using a non-parametric mapping algorithm (MAPMAKER/QTL version 1.9b). Distances between loci were calculated based on the Haldane algorithm. An average spacing of markers of 10 cM was used.

To determine the threshold for suggestive and significant linkage, a permutation test was performed. The permutation test consisted of 5000 random assignments of genotypes with phenotypes. These results confirmed that the LOD thresholds of 2.8 and 4.3 for suggestive and significant linkages, respectively, set by Lander and Kruglyak (Loscalzo et al. (1995) Progress in Cardiovascular Diseases 38:87-104) were appropriate for this study despite the large number of phenotypes tested.

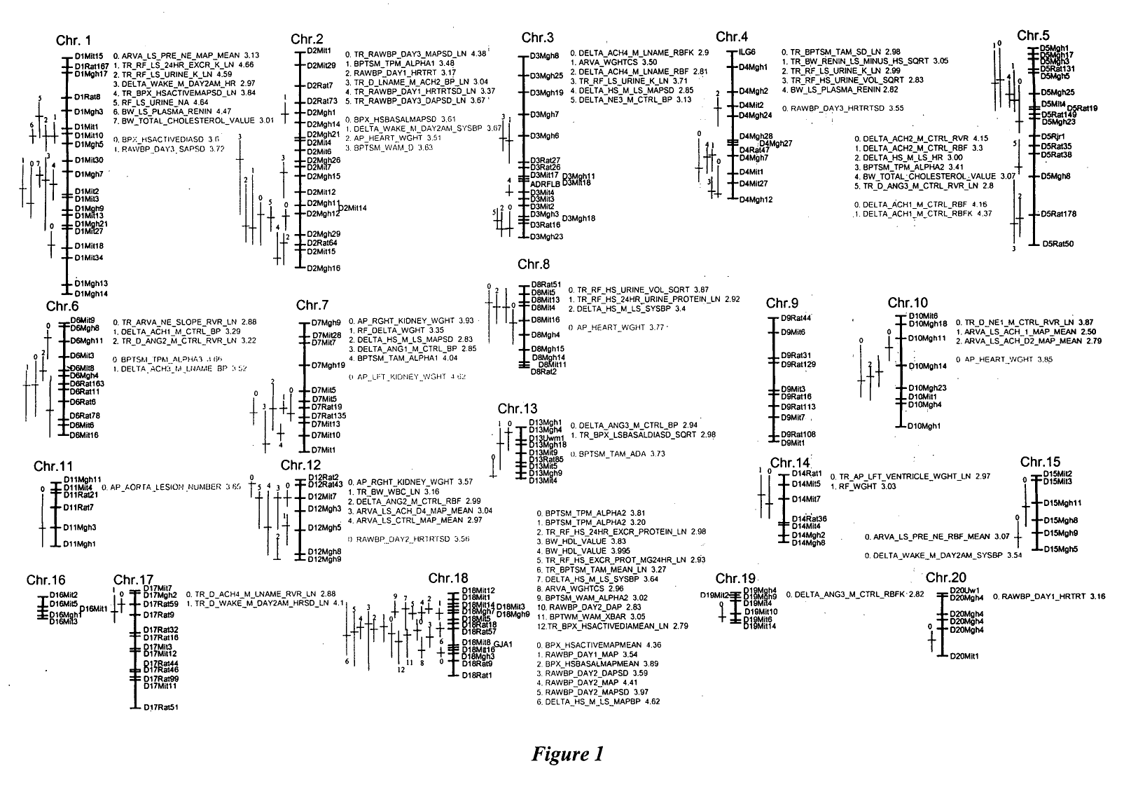

Eighty-one phenotypes had either a parametric LOD score ≧2.8 or non-parametric LOD score ≧3.5; 18 parametric phenotypes had LOD scores between 2.5-2.8; and 26 phenotypes were functionally related to blood pressure. From these 81 parametric and non-parametric phenotypes, 96 QTL were identified of which 69 had an LOD score of >2.8 and 25 had a LOD score of ≧3.5. The 96 QTL identified in the autosomal genome of 113 male progeny from an SS/JrHsd/Mcw×BN/SsNHsd/Mcw intercross are shown in the genetic linkage map of FIG. 1.

### Example 6

### Results of Genetic Linkage Analysis

In general, QTL for blood pressure were clustered in discrete regions on rat chromosomes 1, 2, 3, 7, and 18. These clusters consisted of six or more QTL with overlapping 95% confidence intervals. In four of the five clusters, the determinant phenotypes were independent, indicating that these four clusters represented separate genes rather than a pleiotropic effect. In the fifth cluster, on chromosome 18, significant correlations were found among the determinant phenotypes. These phenotypes could be divided into three functional groups that include phenotypes associated with: vascular reactivity, plasma lipid concentration, and renal function. The clustering of QTL associated with phenotypes belonging to distinct functional groups suggests the presence of a functional cassette as has been observed for QTL in agriculture and biomedical research. (See Thumma B R et al. (2001) J. Exp. Bot. Feb, 52, 203-214; Miner L L, M. R J (1995) Psychopharmacology (Beerl) 117, 62-66; Wakeland et al. (1997) J Clin Immunol 17, 272-281; and Nadeau et al. (2000) Nat. Genet. 25, 381-384).

More specifically, QTL for MAP and RBF responses to ACh were found on chromosome 10 (D10Mgh14); this contributes 17% to the variance of the pressure and RBF in the F2 population. When the contribution of D10Mgh14 to genetic variance was removed using the fix command in MAPMAKER (Lander et al. (1987) Genomics 1:174-181), additional loci on chromosomes 4 (D4Mit2) and 12 (D12Mit7) were found to contribute to the ACh response. The loci on chromosome 10, 4, and 12 were known to harbor genes for nitric oxide synthesis, i.e. NOSIII on chromosome 4, NOSII on chromosome 10, and NOSI on chromosome 12. While increases in the synthesis of nitric oxide mediate much of the vasodilator response to ACh (Loscalzo et al. (1995) Progress in Cardiovascular Diseases 38:87-104), until now, the vasodilator response has not been shown to be associated with all three nitric oxide synthases.

### Example 7

### Development of Physiological Profiling for Studying Physiological Responses

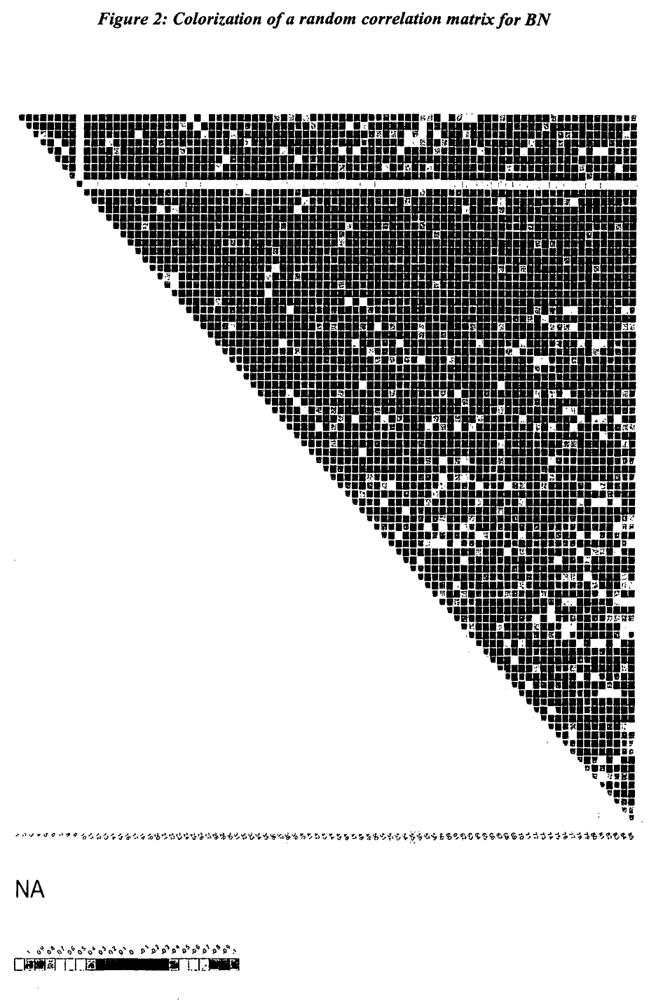

Phenotyping and genetic mapping data obtained as described in Examples 2-6 were used in developing a new analytical strategy—physiological profiling. Genetic mapping of determinant phenotypes associated with hypertension resulted in identification of QTL that, in many cases, were clustered in the same region of the genome. To understand the chromosomal clustering of the phenotypes, a correlation matrix was constructed. The correlation matrix consisted of correlation values determined by linear regression analyses for all pairs of phenotypes. Each correlation value reflected the relationship between two phenotypes. For ease of visual analysis, correlation values ranging from 1 to −1 were presented on the matrix using a color scheme. FIG. 2, for example, is a colorized correlation matrix of the genetically mapped phenotypes of BN rats. Phenotypes were organized in a random order along the top and side of the colorized correlation matrix. To capture the complex interactions among phenotypes, a second analytical procedure was performed to cluster phenotypes into meaningful groups. The clustering procedure was performed using known functional; or physiological relationships (functional clustering). In functional clustering, for example, related phenotypes such as RBF responses to agonists were placed next to each other. FIG. 3 is a functionally clustered correlation matrix, i.e. a physiological profile, consisting of the same phenotypes used in FIG. 2. In the physiological profile depicted in FIG. 3, the phenotypes were clustered based on Guyton's model of blood pressure control (Guyton, A. C. (1972) Monograph).

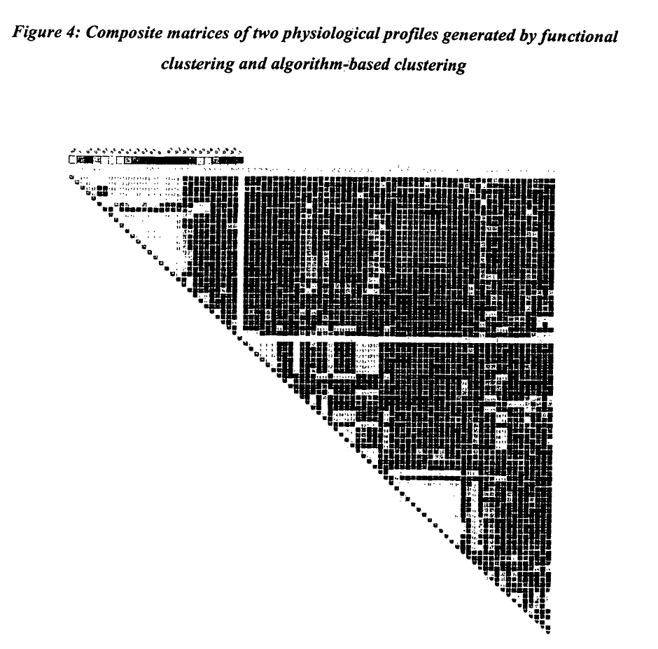

To validate the use of functional clustering, a physiological profile in which phenotypes were clustered using a two-step clustering algorithm that was independent of function was generated. (See, Everitt, B. S. (1993) Cluster Analysis, 3rd Edition, Edward Arnold, Ltd., London, UK; SAS/STAT User's Guide (1990) Version 6, Fourth Edition, Volume 1, pages 519-614; and SAS/STAT User's Guide, 1990, Version 6, Fourth Edition, Vol 2, pages 1614-1631.) The physiological profile generated using the two-step algorithm clustering method was compared with one generated using functional clustering based on Guyton's model of blood pressure control (FIG. 3). The two matrices were overlayed to form a composite profile (FIG. 4). Comparison of the composite profile of FIG. 4 with the physiological profile generated using Guyton's model of blood pressure control (FIG. 3) showed that they were very similar. These results suggest that a correlation matrix generated by functional clustering is useful for establishing a “profile” of physiological function.

### Example 8

### Comparison of the Physiological Profiles of the Parental BN and SS Rats

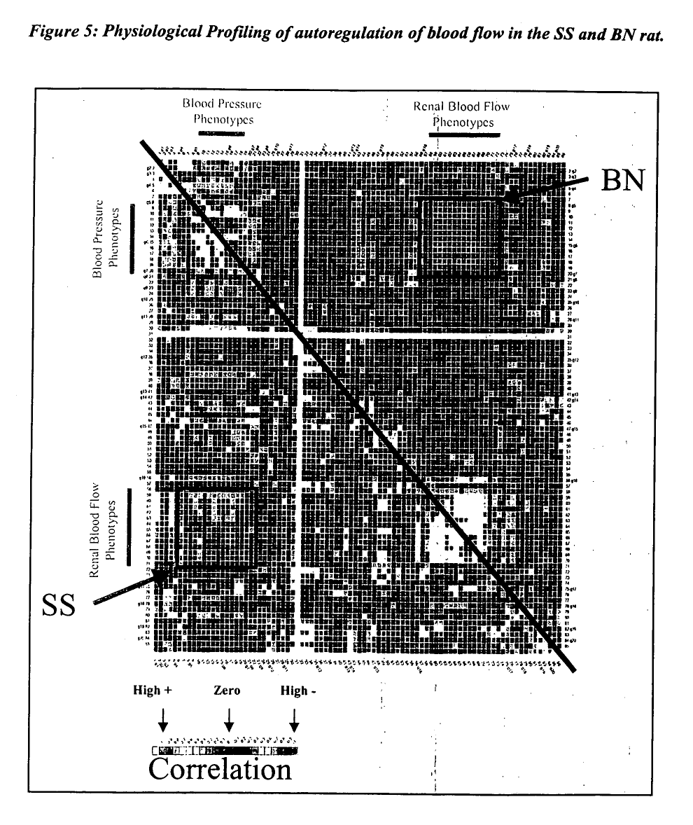

Correlations among phenotypes of the parental BN and SS rats that corresponded to the mapped phenotypes of the F2 intercross were analyzed by physiological profiling. The physiological profiles, shown in FIG. 5, were generated from 50 parental BN rats (top right triangle) and 50 parental SS rats (bottom left triangle). Phenotypes were functionally clustered, i.e. using knowledge of physiological function and without the aid of the correlation matrix. Comparison of the physiological profiles of these two strains shows clear differences in a number of correlations. For example, low positive correlations were observed in the RBF phenotypes of the BN rats, while significant negative correlations among the same phenotypes were observed in the SS rats. These results are consistent with the finding that salt sensitive SS rats cannot regulate renal resistance and blood flow as well as BN rats in response to renal perfusion pressure. The agreement with prior observations illustrates the effectiveness of physiological profiling. Second, the two prominent clusters outlined in red in both strains represent results of dose response curves of acetylcholine, angiotensin II, and norepinephrine. These clusters indicate that both strains have similar physiological responses to these pharmacological agents.

### Example 9

### Comparison of the Physiological Profiles of BN Rats and all F2 Intercross Progeny Rats Generated by Functional and Algorithm Clustering

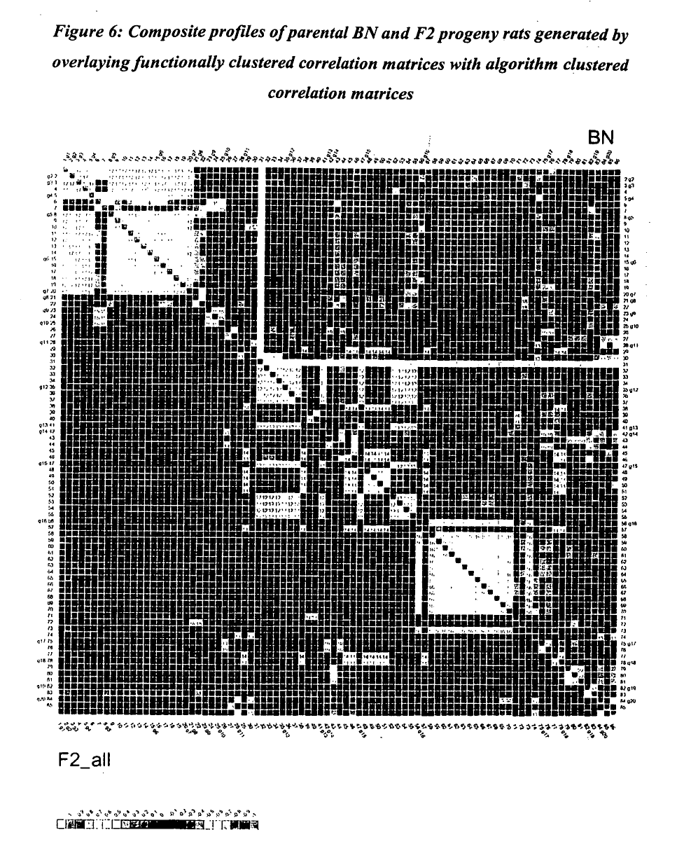

The physiological profile of BN rats was compared with the physiological profile of all F2 intercross progeny (see FIG. 6). Two physiological profiles of the parental BN rats, one generated using functional clustering and the second using a clustering algorithm, were compared by overlaying. Similarly, two physiological profiles of the F2 intercross progeny, one generated using functional clustering and the second using the same clustering algorithm as in Example 7, were compared by overlaying. The resulting composite profiles of the BN rats (top right and above diagonal) and the F2 progeny rats (bottom left and below the diagonal) are shown in FIG. 6. A blending of profiles in the F2 intercross progeny was observed when the composite BN and F2 progeny profiles (FIG. 6) and the SS profile in FIG. 5 were compared. Although functional clustering and clustering by purely statistical methods yielded similar results as indicated by the composite profile, the physiological profile generated by statistical methods revealed two clusters of traits (clusters 12 and 14) that were not known before.

### Example 10

### Effects of Allelic Substitution on the Physiological Profiles

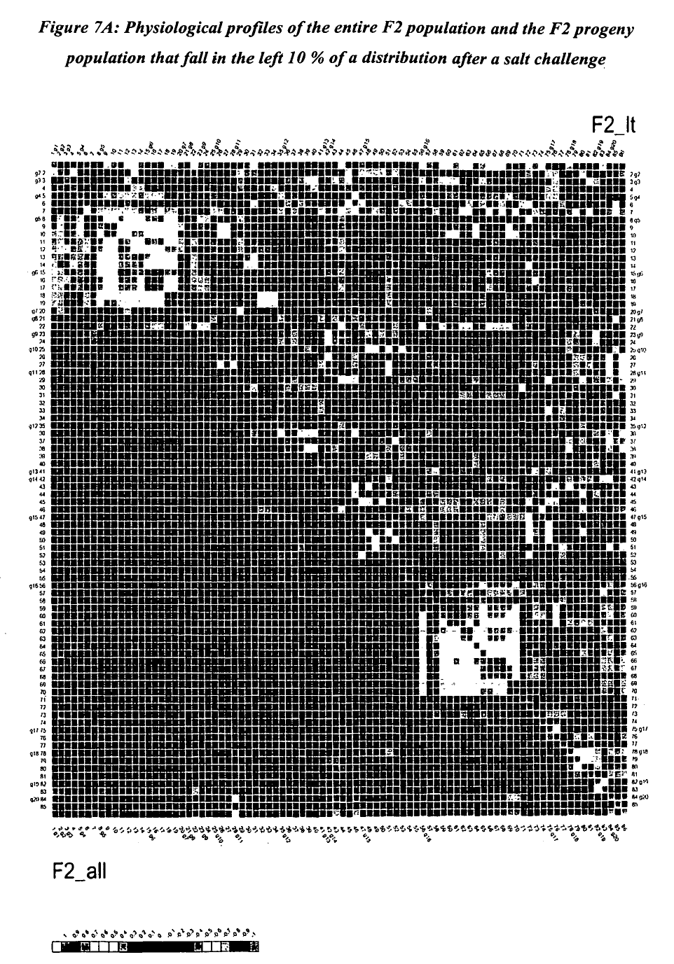

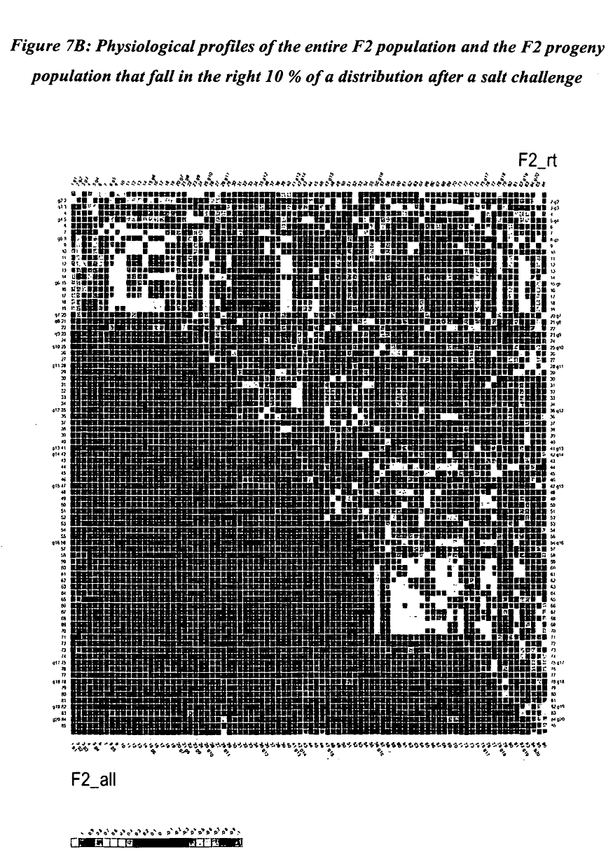

Physiological profiling was used to assess how a group of F2 animals respond to a salt challenge. FIGS. 7A and 7B are comparisons of the physiological profiles of the entire F2 population with the F2 animals that have QTL that protect against a salt load. The F2 animals that were protected against a salt load were those that fell into the 10% tails of a distribution after a salt challenge. In general, low correlations between phenotypes were observed in the physiological profile of the entire F2 population. In contrast, strong positive correlations between some phenotypes were observed in the physiological profile of animals that were protected against a salt challenge. Therefore, physiological profiling is effective in capturing differences in physiology between distinct groups.

### Example 11

### Physiological Profiling of Blood Pressure Determinant Phenotypes

Since genetic mapping results of Example 6 demonstrated that the vasodilator response to Ach is associated with loci containing three nitric oxide synthases, the impact of BN and SS alleles of all three NOS genes on the mapped phenotypes was examined by physiological profiling. This also allowed for assessing the systems biology of the other mapped cardiovascular phenotypes.

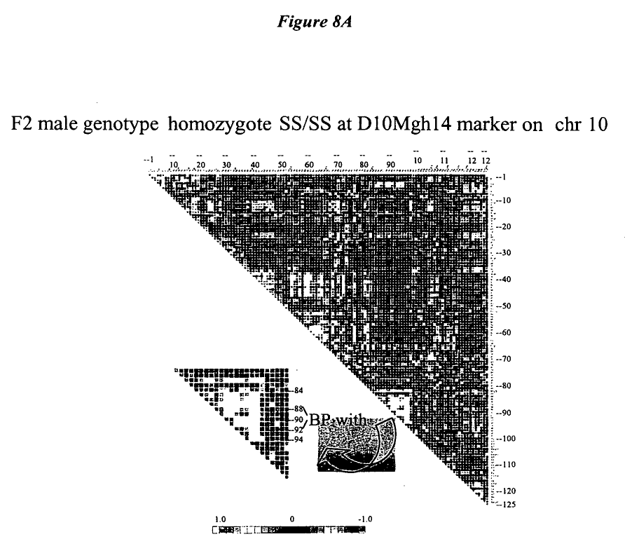

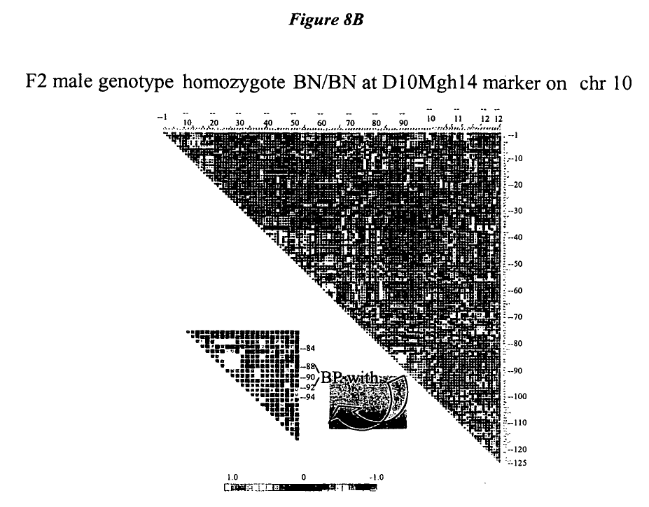

The mapped phenotypes were analyzed by physiological profiling using functional clustering based on Guyton's model of blood pressure control. FIG. 8A is the physiological profile for F2 male rats that were homozygous SS for D10Mgh14 (the flanking marker for NOSII), and FIG. 8B is the physiological profile for F2 male rats that were homozygous BN for D10Mgh14. The correlation patterns were found to be quite different when the SS and BN profiles were compared. In F2 rats homozygous for the SS allele at D10Mgh14 (NOSII), positive correlations were observed among blood pressures determined immediately before, during, and after the short-term intravenous administration of norepinephrine (NE), angiotensin II (Ang II), and acetylcholine (see FIG. 8A, cells #85-94). In contrast, F2 rats homozygous for the BN allele at D10Mgh14 (NOSII) exhibited weak correlations among the same phenotypes (see FIG. 8B). Furthermore, the relationships of measured differences in systolic, diastolic, and mean arterial pressures before and following infusions of NE (cells #88-92) also were different.

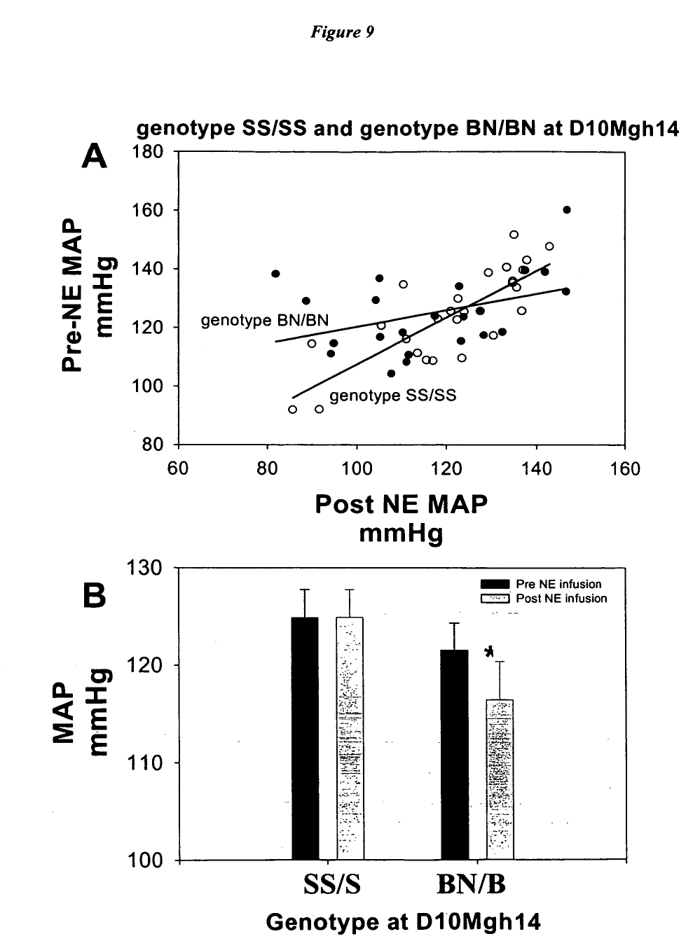

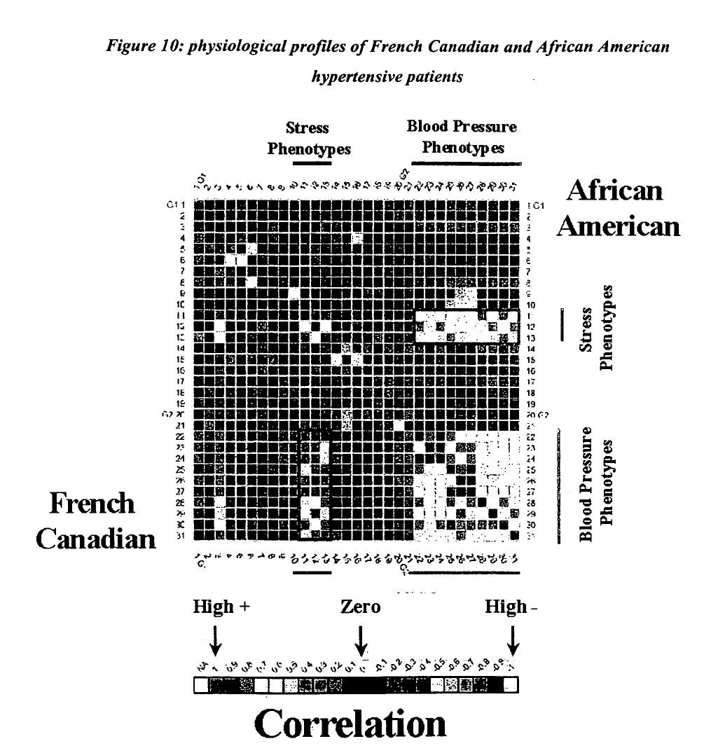

The relationship between MAP before and after infusion of NE in F2 rats carrying the BN or SS allele at D10Mgh14 (NOSII) is further illustrated in FIGS. 9A and 9B. FIG. 9A is a graph demonstrating the correlation between MAP before and after infusion of NE in F2 rats carrying the BN allele (closed circles) and in F2 rats carrying the SS allele (open circle) at D10Mgh14 (NOSII). Although the F2 rats carrying the BN allele (closed circles) at D10Mgh14 exhibited a lack of correlation between arterial pressure levels before and following intravenous infusions of NE, a significant positive correlation was observed in F2 rats homozygous for the SS allele (open circles) at NOSII. FIG. 9B is a bar graph summarizing the average levels of MAP before (solid bars) and following completion (open bars) of intravenous infusions, of three doses of norepinephrine in male rats carrying the SS or BN allele at NOSII. Arterial pressures of male rats carrying the SS allele at NOSII returned precisely to control levels, while the arterial pressures of rats carrying the BN allele fell significantly below control and remained at hypotensive levels for as long as 10 minutes. Although it has been reported that NO plays a role in the control of vascular tone and blood pressure, the finding that NOS is involved in vasoconstrictor agents-induced hypotension has not been reported.

The physiological profile of the heterozygote at D10Mgh14 exhibited a correlation pattern that was intermediate between SS and BN, although closer to BN (data not shown).

Physiological profiles for rats partitioned by alleles on chromosome 4 at D4Mit2 (NOSIII) demonstrated similar relationships for these traits (#85-94) for SS versus BN alleles (see http://brc.mcw.edu/phyprf). The similarity in physiological profiles of rats partitioned by at NOSIII and those of rats partitioned at NOSII indicate similar gene effects on overall physiology.

### Example 12

### Other Novel Relationships Derived from Physiological Profiling

Positive correlations were observed between the urinary excretion of protein and plasma lipid concentration (FIGS. 8A and 8B, cells 115 vs. 34), and between kidney weight and plasma lipid concentrations (FIGS. 8A and 8B, cells 115 vs. 36) in F2 rats homozygous BN for the NOSII allele. Although hyperlipidemia, proteinuria, and renal hypertrophy are related symptoms seen in hypertensive and diabetic nephropathy and end stage renal disease, the influence of a NOSII genotype on the relationships between these indices of renal end organ damage and hyperlipidemia has not been described previously. Thus, the present finding that the severity of proteinuria, renal hypertrophy, and hyperlipidemia in an F2 population of rats is influenced by the allelic variations of inducible NOS (NOSII) gene is novel and creates new directions for further research.

Another relationship determined by physiological profiling that was not detected by linkage analysis was the strong positive correlation found between Angiotensin II-induced reductions of RBF and the chronic urinary excretion of protein in rats homozygous BN at the D13Mgh18 allele. This finding contrasts with the significant negative correlation observed in F2 homozygous SS rats at this same allele. (Urine protein excretion was determined during steady-state conditions of high salt intake, while renal blood responses to AngII were determined following a day of salt depletion in anesthetized F2 rats using three doses of AngII.) These results provide a genetic basis for results of many clinical studies of essential hypertension in which patients have been stratified based on their renal vascular responses to AngII as “modulators” or “nonmodulators” (Hollenberg et al. (1978) Medicine 57:167-178). In 40-50 percent of the essential hypertensive population, adrenal and renal vascular responses to AngII are not modified by changes in sodium intake as would be expected. These individuals have been called “non-modulators” (Hollenberg et al. (1978) Medicine 57:167-178). It has been documented that non-modulators exhibit a higher percentage of one or both parents with hypertension suggesting that this renal abnormality is inherited and linked to the development of hypertension (see Hollenberg et al. (1978) Medicine 57:167-178). In the present study, SS fed a high salt diet exhibited substantial proteinuria compared to BN parental rats. The physiological profile revealed that in F2 rats, it was possible to predict the renal blood flow AngII sensitivity based on genotype and protein excretion levels. A narrow region on rat chromosome 13 near D13Mgh18 enables a prediction of modulators and non-modulators using individual genotype. The only identified gene that maps very closely to D13Mgh18 is renin, an obvious candidate for these responses. The present result provides a genetic basis for modulators and nonmodulators that could be explored further in a genetic rat model of hypertension.

### Example 13

### Physiological Profiles of African a American and French Canadian Patients with Hypertension

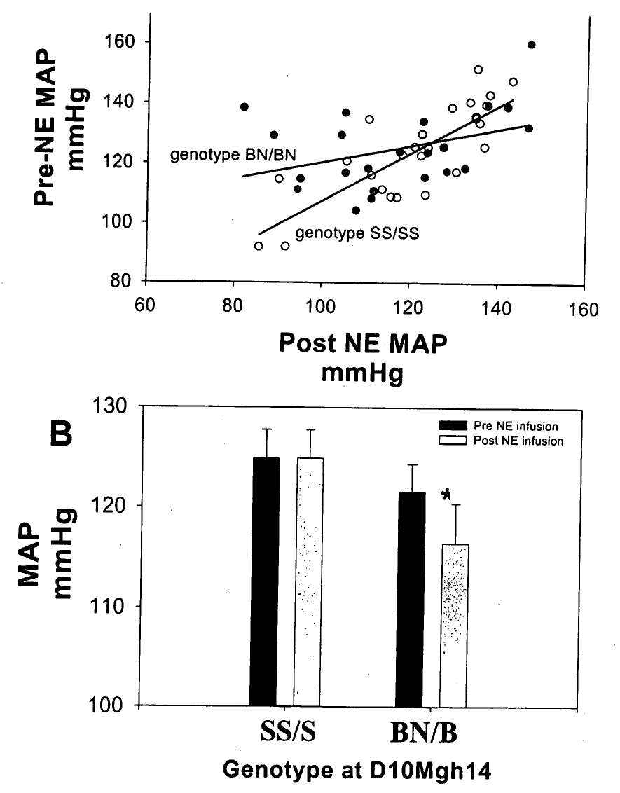

Physiological profiling was used to assess correlations between phenotypes associated with blood pressure in resting and stressed patients with hypertension. Patients were African American and French Canadian sibling pairs with hypertension. Patients underwent an extensive 2-day in-house protocol (see Kotchen et al. (2000) Hypertension 36:7-13 and Pausova et al. (2001) Hypertension 38:41-47) at the Medical College of Wisconsin or Centre de Recherche, Centre Hospitalier de l'Universite de Montreal (CHUM), Montreal, Canada. FIG. 10 depicts the physiological profiles generated for the French Canadian and the African American patient populations. A comparison of the physiological profiles of the two patient cohorts revealed that the sibling-sibling profiles were more similar than the matrices generated when only one of the siblings was used. These data indicate that physiological profiling is useful for comparing heritable traits.

Therefore, physiological profiling is a powerful means to (1) summarize the complex physiological interactions into a single image, (2) capture graphically in a single image the numerous differences in the cardiovascular system of different rats strains, and (3) identify physiological characteristics not evident by genetic mapping data.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-11. (canceled)

12. A method of partitioning organisms into homogeneic subclasses, said method comprising comparing the physiological profiles of said organisms and partitioning said organisms into homogeneic subclasses based on differences in said physiological profiles.

13. The method of claim 12, wherein said organisms exhibit multifactorial disease condition.

14. The method of claim 12, further comprising performing expression profiling and further partitioning said organisms into said homogeneic subclasses based on said expression profiling.

15. A method of assigning an organisms to a homogeneic subclass of organisms, said method comprising generating a physiological profile of said organism and identifying said organism as belonging to said homogeneic subclass based on said physiological profile.

16. The method of claim 15, wherein said homogeneic subclass of organisms exhibits a multifactorial disease condition.

17-18. (canceled)

19. A computer-readable medium comprising a physiological profile.

20. The computer-readable medium of claim 19, wherein said physiological profile comprises a plurality of colors, wherein each color indicates a selected degree of correlation.

21-25. (canceled)

26. A method for modifying or supplementing actuarial tables for life and health insurance comprising
(a) identifying homogeneic subclasses of humans according to the method of claim 12, and
(b) modifying or supplementing said actuarial tables based on said identified homogeneic subclasses.
